SEQUENCE LISTING (8 sequences)

SEQ ID NO: 1 - DNA, 46 bp, Artificial sequence
  Synthetic
tttttgggctaacaggaggaattaaccatggcgcatcattggggtt

SEQ ID NO: 2 - DNA, 46 bp, Artificial sequence
  Synthetic
tcttctctcatccgccaaaacagccaagcttttagtgatggtggtg

SEQ ID NO: 3 - DNA, 26 bp, Artificial sequence
  Synthetic
gtctgctgccgtagagcctggattac

SEQ ID NO: 4 - DNA, 26 bp, Artificial sequence
  Synthetic
gtaatccaggctctacggcagcagac

SEQ ID NO: 5 - DNA, 29 bp, Artificial sequence
  Synthetic
gaatttcaatggttagggcgagccggaag

SEQ ID NO: 6 - DNA, 29 bp, Artificial sequence
  Synthetic
cttccggctcgccctaaccattgaaattc

SEQ ID NO: 7 - DNA, 26 bp, Artificial sequence
  Synthetic
ggcacaaagattagccaattgcgaag

SEQ ID NO: 8 - DNA, 26 bp, Artificial sequence
  Synthetic
cttcgcaattggctaatctttgtgcc

(19) United States
(12) Patent Application Publication
(10) Pub. No.: US 2021/0072238 A1
(43) Pub. Date: Mar. 11, 2021

(54) IMMOBILIZATION OF PROTEINS WITH CONTROLLED ORIENTATION AND LOAD

(71) Applicant: Oregon State University, Corvallis, OR (US)

(72) Inventors: Ryan A. Mehl, Corvallis, OR (US); Riley Matthew Bednar, Corvallis, OR (US)

(73) Assignee: Oregon State University, Corvallis, OR (US)

(21) Appl. No.: 17/016,032

(22) Filed: Sep. 9, 2020

Related U.S. Application Data
(60) Provisional application No. 62/897,821, filed on Sep. 9, 2019.

(51) Int. Cl. G01N 33/543; C07K 1/04; C12N 11/14

(52) U.S. Cl. CPC G01N 33/54353; C12N 11/14; C07K 1/042

(57) ABSTRACT

Methods for immobilizing a protein or functional protein fragment on a surface in a controlled orientation, for immobilizing a protein or functional protein fragment on a surface with efficient immobilization loading of the protein or protein fragment, and for immobilizing a protein or functional protein fragment on a surface with retention of the activity of the protein or protein fragment. In the methods, a tetrazine-modified protein or a tetrazine-modified functional protein fragment is contacted with a trans-cyclooctene-modified surface to provide a surface having the protein or functional protein fragment immobilized thereon. Surfaces having a protein or functional protein fragment immobilized thereon obtainable by the method and methods for using the surfaces for measuring the binding of a ligand to a protein or functional protein fragment.

### CROSS-REFERENCE TO RELATED APPLICATION

This application claims the benefit of U.S. Application No. 62/897,821, filed Sep. 9, 2019, expressly incorporated herein by reference in its entirety.

### STATEMENT OF GOVERNMENT LICENSE RIGHTS

This invention was made with Government support under MCB 1518265 awarded by the National Science Foundation. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 72346_Seq_Final_2020-08-28.txt. The text file is 1.71 KB; was created on Aug. 28, 2020; and is being submitted via EFS-Web with the filing of the specification.

### BACKGROUND OF THE INVENTION

An unmet challenge in the field of material science is the covalent immobilization of proteins on surfaces in defined loads and orientations with minimal loss in function and nonspecific adsorption. In 1951, Campbell and colleagues developed the first covalent immobilization strategy while optimizing antibody purification; however, this approach did not allow for the control of orientation. Over the next three decades, the field evolved to make immobilized protein surfaces more homogenous in orientation through attachment via unique cysteine residues and affinity binding proteins. While not generalizable, these approaches revealed that immobilized protein orientation can affect protein stability and activity. The advent of genetic code expansion (GCE) allowed site-specific incorporation of bioorthogonal reactive groups into proteins, thereby providing a generalizable approach to controlling protein orientation.

Despite these advances in controlling orientation, no general technology has been developed to control protein loading on surfaces, making it challenging to precisely define how protein activity and stability are impacted by surface functionality or protein quantity, orientation, and density. Even though there are techniques to directly assess the total amount of immobilized protein (e.g., radiolabeling and surface-sensitive techniques, such as X-ray photoelectron spectroscopy), it is difficult to define what fraction of the immobilized protein is non-specifically immobilized, impaired, or denatured. Commonly used indirect approaches, such as solution depletion, are less informative since in addition to the aforementioned limitations, they are easily convoluted by off-target, non-specific adsorption to the containment vessel. In addition, solution depletion methods are not applicable to flat surfaces or small scales such as those used in lab-on-a-chip applications.

The vast combinations of material surfaces and proteins available adds to the challenge of precise and reliable immobilization of defined amounts of active, site-specifically-oriented proteins. To minimize non-specific adsorption, others have focused on optimization of solution parameters and passivation of the surface with antifouling coatings such as polyethylene glycol (PEG) polymers. However, the high protein concentrations and lengthy incubation times required for immobilization by sluggish reactions routinely leads to some degree of non-specific adsorption or aggregation.

Despite the advances in surface immobilization for proteins noted above, a need exists for controlling protein load, protein activity once loaded, and the orientation of protein immobilized on a surface. The present invention seeks to fulfill this need and provides further related advantages.

### SUMMARY OF THE INVENTION

The present invention provides methods for immobilizing a protein or functional protein fragment on a surface in a controlled orientation, for immobilizing a protein or functional protein fragment on a surface with efficient immobilization loading of the protein or protein fragment, and for immobilizing a protein or functional protein fragment on a surface with retention of the activity of the protein or protein fragment, surfaces having a protein or functional protein fragment immobilized thereon, and methods for using the surfaces for measuring the binding of a ligand to a protein or functional protein fragment.

In one aspect, the invention provides a method for immobilizing a protein or functional protein fragment on a surface in a controlled orientation. In certain embodiments, the method comprises contacting a tetrazine-modified protein or a tetrazine-modified functional protein fragment with a trans-cyclooctene-modified surface to provide a surface having the protein or functional protein fragment immobilized thereon, wherein the tetrazine-modified protein or the tetrazine-modified functional protein fragment has been genetically encoded to include a tetrazine moiety at a predetermined amino acid site, wherein the tetrazine-modified protein or tetrazine-modified functional protein fragment is prepared by genetic encoding using a non-canonical amino acid bearing a tetrazine moiety represented by the formula (I):

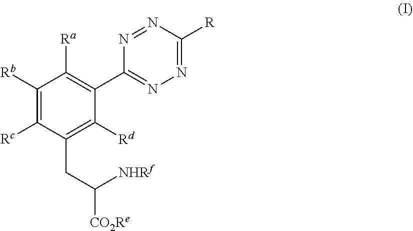

or a stereoisomer or salt thereof, wherein

R is selected from substituted or unsubstituted C1-C6 alkyl group and substituted or unsubstituted phenyl group;

R^{a}, R^{b}, R^{c}, and R^{d} are independently selected from hydrogen, C1-C3 alkyl, C1-C3 haloalkyl, C1-C3 alkoxy, and halo;

R^{e} is hydrogen, a counter ion, or a carboxyl protecting group; and

R^{f} is hydrogen or an amine protecting group.

In another aspect, the invention provides a method for efficiently immobilizing a protein or functional protein fragment on a surface. In certain embodiments, the method comprises contacting a tetrazine-modified protein or a tetrazine-modified functional protein fragment with a trans-cyclooctene-modified surface to provide a surface having the protein or functional protein fragment immobilized thereon, wherein contacting the tetrazine-modified protein or the tetrazine-modified functional protein fragment with a trans-cyclooctene-modified surface comprises contacting a pre-determined amount of the protein or functional protein fragment and the amount of protein or functional protein fragment immobilized on the surface is at least about 80 percent of the pre-determined amount of the protein or functional protein fragment contacted with the surface.

In a further aspect, the invention provides a method for immobilizing a protein or functional protein fragment on a surface with retention of the activity of the protein or protein fragment, comprising contacting a tetrazine-modified protein or a tetrazine-modified functional protein fragment with a trans-cyclooctene-modified surface to provide a surface having the protein or functional protein fragment immobilized thereon, wherein the protein or functional protein fragment immobilized on the surface retains at least about 80 percent of the activity of the tetrazine-modified protein or functional protein fragment.

In another aspect, the invention provides a surface having a protein or functional protein fragment immobilized thereon obtainable by the methods of the invention.

In a further aspect, the invention provides a method for measuring the binding of a ligand to a protein or functional protein fragment using the surface.

### DESCRIPTION OF THE DRAWINGS

The patent or application file contains at least one drawing executed in color. Copies of this patent or patent application publication with color drawings will be provided by the Office upon request and payment of the necessary fee.

The foregoing aspects and many of the attendant advantages of this invention will become more readily appreciated as the same become better understood by reference to the following detailed description, when taken in conjunction with the accompanying drawings.

FIGS. 1A-1D illustrate the protein-limited immobilization of oriented representative proteins (tsCA-Tet2.0 proteins) onto a representative functionalized surface (sTCO-functionalized surface). FIG. 1A depicts the inverse electron demand Diels-Alder reaction between Tet2.0 and sTCO and its relevant characteristics. FIG. 1B illustrates the process of tsCA-Tet2.0 protein production and immobilization onto sTCO surfaces. FIG. 1C illustrates representative orientations of the three tsCA (cyan) variant's active-sites relative to the surface. FIG. 1D illustrates the concept of protein-limited control of loading (e.g., 25, 50, and 100%).

FIGS. 2A-2C graphically illustrates the characterization of tsCA variants, sTCO-beads, and the immobilization process. FIG. 2A compares relative enzymatic activity of representative tsCA variants before and after conjugation with sTCO-PEG₅₀₀₀. FIG. 2B compares titration monitoring the depletion of tsCA₁₈₆ enzyme activity from post-reaction supernatant with increasing amounts of sTCO-beads. Intersection of best-fit lines of the first-four and last-two points represents a protein binding capacity of approximately 120 ng tsCA per mg of beads. FIG. 2C compares partitioning of enzymatic activity between sTCO-beads and supernatant after immobilization of tsCA_{WT} and tsCA₁₈₆ on various bead-types. “Conjugated” (panel A) and “Blocked Protein” (panel C, condition iv) tsCA_{WT} are tsCA_{WT} that were exposed to sTCO-PEG₅₀₀₀ identically to their tsCA_{Tet2.0} counterpart in each experiment. Because tsCA_{WT} lacks a Tet2.0 moiety, the protein remains unmodified, but serves as a control that has undergone the same treatment as tsCA_{Tet2.0} proteins in each instance. Bead-associated activities throughout this report are corrected for bead-loss during washes.

FIGS. 3A-3C illustrate structural characterization of Tet2.0 incorporated into tsCA. FIG. 3A (A) is an overlay of tsCA_{WT} (teal; PDB code 6B00), tsCA₁₈₆ (cyan), and tsCA₂₃₃ (lime) with incorporated Tet2.0 and zinc (grey sphere) with ligating histidines shown. FIG. 3B illustrate views of 2Fₒ-F_{c} electron density (1.0*ρᵣₘₛ) for Tet2.0 in (i) tsCA₁₈₆ (cyan) and (ii) tsCA₂₃₃ (lime). For both, right view is rotated by ˜90° with respect to the left view. FIG. 3C shows the shift in Tet2.0 position upon reaction with sTCO in crystallo. Overlay of Tet2.0 in tsCA₁₈₆ before (cyan) and after in crystallo reaction with sTCO (seafoam), showing a shift (direction indicated by arrow) between terminal methyl groups in Tet2.0 position upon reaction with sTCO.

FIGS. 4A-4D graphically illustrate protein-limited immobilization of oriented tsCA_{Tet2.0} onto sTCO-beads. FIG. 4A compares relative enzymatic and radioactivity of pre-immobilization (Free), supernatant, and bead-associated fractions from tsCA₂₃₃ immobilized at 100%, 50%, and 25% loads. Values are relative to the Free-100% load. Internally-normalized values from a tsCA_{WT}-100% load are also shown. FIG. 4B compares plots as a function of the load level showing best fit lines for each enzymatic-activity, radioactivity (from FIG. 4A), and XPS N₁ₛ signal arising from post-immobilization beads exposed to various loads of tsCA₂₃₃. FIG. 4C compares relative enzymatic activity of pre-immobilization (Free), supernatant, and bead-associated fractions of all tsCA_{Tet2.0} variants immobilized at 100%, 50%, and 25% loads, as in FIG. 4A. FIG. 4D compares relative enzyme-activity of all tsCA_{Tet2.0} variants at 100%-load relative to tsCA_{WT} (n=6). Significant differences at the p<0.05 (*) and p<0.01(**) as determined by a heteroskedastic, two-tailed t-test are shown.

FIGS. 5A-5D illustrates protein-limited immobilization of sfGFP₁₅₀ onto sTCO-SAMs. FIG. 5A is an overview of sTCO-SAM preparation and sfGFP₁₅₀ immobilization. FIG. 5B shows representative TIRF-microscopy images of immobilized sfGFP₁₅₀ at six loads (0, 0.3, 0.5, 0.7, 1.0, 3.0 μM). FIG. 5C compares relative fluorescence of immobilized sfGFP₁₅₀ as a function of load (n=3 spots, with 3 images per spot). Best-fit lines for first-five points and last-four points shown. FIG. 5D compares relative fluorescence of various sTCO-SAMs after immobilization of sfGFP₁₅₀. “Blocked-SAM” surface was pre-reacted with tetrazine-PEG₅₀₀₀ before immobilization, while “Blocked-Protein” represents sTCO-SAMs exposed to sfGFP₁₅₀ pre-reacted with sTCO-PEG₅₀₀₀.

FIG. 6 is a schematic illustration of the synthesis of 4-nitrophenyl active ester of trans-cyclooctene (sTCO) and disulfide attached di-sTCO derivatives.

FIGS. 7A and 7B illustrate pre-determined immobilization of sfGFP-N150[Tet2.0] and sfGFP-N150[Tet3.0] on sTCO-Sepharose resin. FIG. 7A illustrates relative fluorescence of free protein (equivalent to the amount of protein applied to the resin), and the resulting supernatant after a 5-minute exposure to sTCO-sepharose. FIG. 7B illustrates relative fluorescence of sTCO-sepharose, and the resulting supernatant after a 5-minute reaction period.

### DETAILED DESCRIPTION OF THE INVENTION

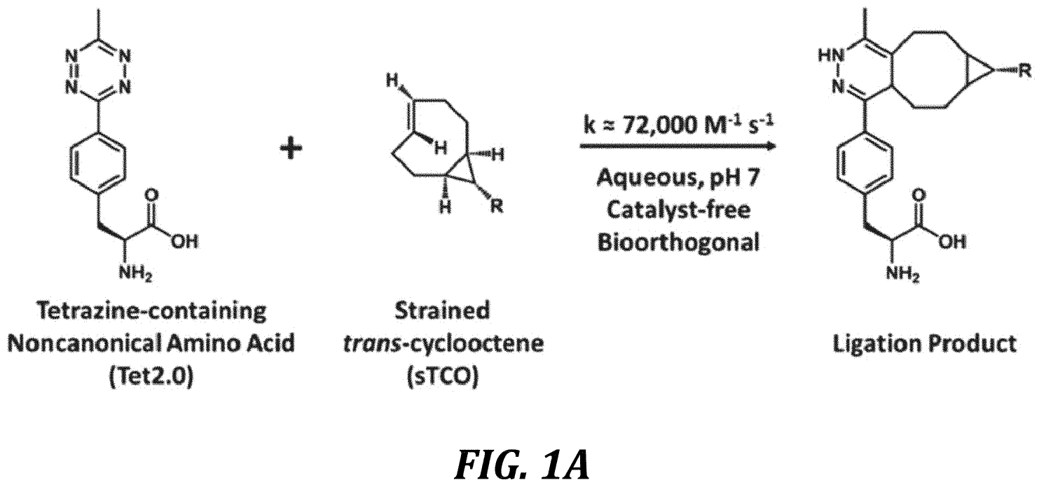

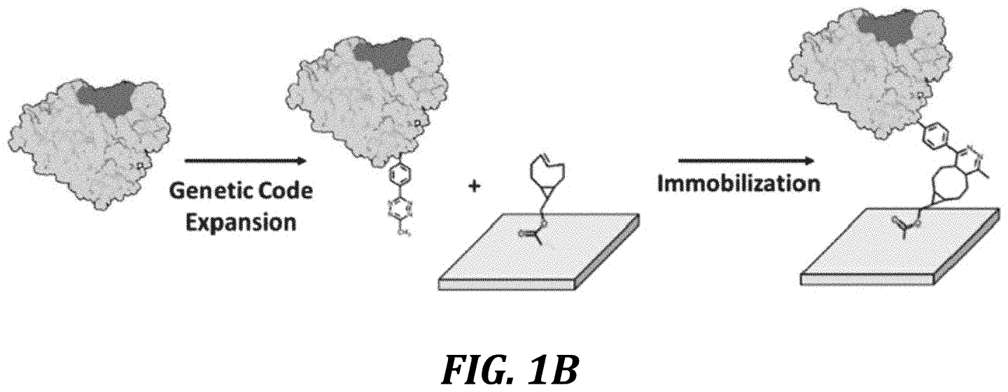

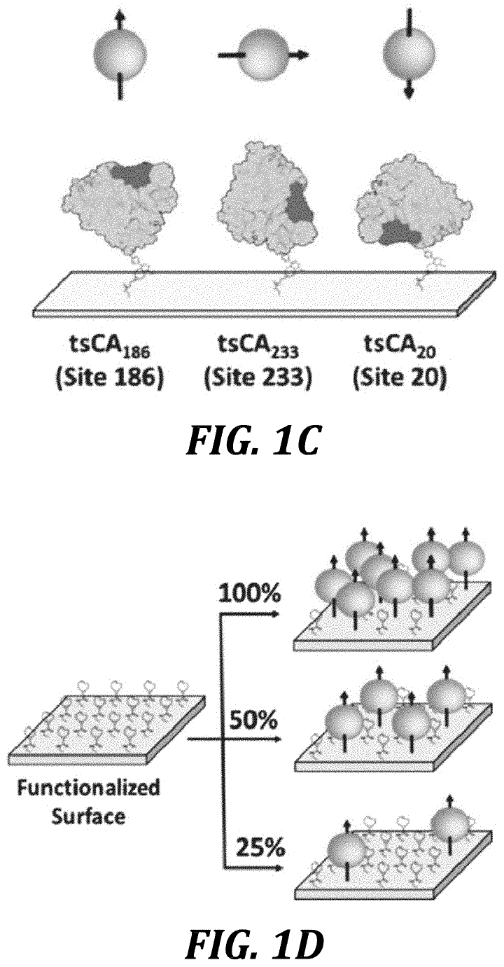

The present invention provides an alternative to conventional techniques for controlling protein load. In the practice of the present invention, optimization of the immobilization reaction itself may serve to control protein load. In the protein immobilization methods of the invention, the rate of the immobilization reaction is sufficiently great such that the protein concentration and incubation time can be markedly reduced allowing for the protein to serve as a limiting reagent. Moreover, the present immobilization reaction avoids harsh reaction conditions and side reactions and favor the preservation of protein activity. The immobilization reaction enables pre-specified, sub-monolayer loading based solely on the amount of protein applied (i.e., “protein-limited immobilization,” see FIG. 1D). The reaction achieves rapid protein-limited immobilization by removing protein from solution and therefore outcompetes protein denaturation, surface fouling, and non-specific adsorption. This protein-limited immobilization approach allows a general method for specific, rapid, gentle, and quantitative immobilization of proteins to a material surface while preserving protein function.

In one aspect, the invention provides a method for immobilizing a protein or functional protein fragment on a surface in a controlled orientation. In certain embodiments, the method comprises contacting a tetrazine-modified protein or a tetrazine-modified functional protein fragment with a trans-cyclooctene-modified surface to provide a surface having the protein or functional protein fragment immobilized thereon, wherein the tetrazine-modified protein or the tetrazine-modified functional protein fragment has been genetically encoded to include a tetrazine moiety at a predetermined amino acid site, wherein the tetrazine-modified protein or tetrazine-modified functional protein fragment is prepared by genetic encoding using a non-canonical amino acid bearing a tetrazine moiety represented by the formula (I):

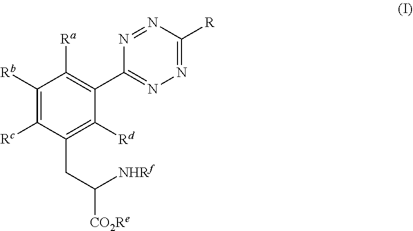

or a stereoisomer or salt thereof, wherein

R is selected from substituted or unsubstituted C1-C6 alkyl group and substituted or unsubstituted phenyl group;

R^{a}, R^{b}, R^{c}, and R^{d} are independently selected from hydrogen, C1-C3 alkyl, C1-C3 haloalkyl, C1-C3 alkoxy, and halo;

R^{e} is hydrogen, a counter ion, or a carboxyl protecting group; and

R^{f} is hydrogen or an amine protecting group.

In certain embodiments, R is selected from substituted or unsubstituted C1-C6 alkyl group; R^{a}, R^{b}, R^{c}, and R^{d} are hydrogen; R^{e} is hydrogen, a counter ion, or a carboxyl protecting group; and R^{f} is hydrogen or an amine protecting group.

In the method, the tetrazine-modified protein or tetrazine-modified functional protein fragment is prepared by genetic encoding using a non-canonical amino acid bearing a tetrazine moiety such as 3-(6-methyl-s-tetrazin-3-yl)phenylalanine (Tet-v3.0-methyl), 3-(6-ethyl-s-tetrazin-3-yl)phenylalanine (Tet-v3.0-ethyl), 3-(6-isopropyl-s-tetrazin-3-yl)phenylalanine (Tet-v3.0-isopropyl), 3-(6-t-butyl-s-tetrazin-3-yl)phenylalanine (Tet-v3.0-t-butyl), or 3-(6-n-butyl-s-tetrazin-3-yl)phenylalanine (Tet-v3.0-n-butyl).

The phrase “genetically encoded to include a tetrazine moiety at a predetermined amino acid site” refers to the process described herein by which a non-canonical amino acid bearing a tetrazine moiety is selectively incorporated into a protein or a functional protein fragment to provide a tetrazine-modified protein or a tetrazine-modified functional protein fragment at an amino acid selected for modification. The genetic encoding method described herein can be used to incorporate a non-canonical amino acid bearing a tetrazine moiety at any site (i.e., amino acid position) in the protein or a functional protein fragment. By virtue of the position of the tetrazine moiety in the tetrazine-modified protein or the tetrazine-modified functional protein fragment, and because of the selective reactivity of the tetrazine moiety with the trans-cyclooctene-modified surface, the orientation of the protein or functional protein fragment on the surface is controlled. The method allows for control of the presentation of the protein or functional protein fragment on the surface.

In another aspect, the invention provides a method for efficiently immobilizing a protein or functional protein fragment on a surface. In certain embodiments, the method comprises contacting a tetrazine-modified protein or a tetrazine-modified functional protein fragment with a trans-cyclooctene-modified surface to provide a surface having the protein or functional protein fragment immobilized thereon, wherein contacting the tetrazine-modified protein or the tetrazine-modified functional protein fragment with a trans-cyclooctene-modified surface comprises contacting a pre-determined amount of the protein or functional protein fragment and the amount of protein or functional protein fragment immobilized on the surface is at least about 80 percent of the pre-determined amount of the protein or functional protein fragment contacted with the surface.

In a further aspect, the invention provides a method for immobilizing a protein or functional protein fragment on a surface with retention of the activity of the protein or protein fragment, comprising contacting a tetrazine-modified protein or a tetrazine-modified functional protein fragment with a trans-cyclooctene-modified surface to provide a surface having the protein or functional protein fragment immobilized thereon, wherein the protein or functional protein fragment immobilized on the surface retains at least about 80 percent of the activity of the tetrazine-modified protein or functional protein fragment.

In certain of these embodiments, contacting a tetrazine-modified protein or tetrazine-modified functional protein fragment with a trans-cyclooctene-modified surface comprises contacting a pre-determined amount of the protein or functional protein fragment and the amount of protein or functional protein fragment immobilized on the surface is about 90 percent, or about 100 percent, of the pre-determined amount of the protein or functional protein fragment contacted with the surface.

In certain of these embodiments, the protein or functional protein fragment immobilized on the surface retains about 100 percent of the activity of the tetrazine-modified protein or functional protein fragment.

In the above methods, contacting a tetrazine-modified protein or functional protein fragment with a trans-cyclooctene-modified surface comprises contacting a pre-determined amount of the protein or functional protein fragment and the amount of protein or functional protein fragment immobilized on the surface is at least about 80 percent of the pre-determined amount of the protein or functional protein fragment contacted with the surface.

In certain embodiments, contacting a tetrazine-modified protein or tetrazine-modified functional protein fragment with a trans-cyclooctene-modified surface comprises contacting a pre-determined amount of the protein or functional protein fragment and the amount of protein or functional protein fragment immobilized on the surface is about 90%, or about 100 percent, of the pre-determined amount of the protein or functional protein fragment contacted with the surface.

In the above methods, the protein or functional protein fragment immobilized on the surface retains at least about 80 percent of the activity of the tetrazine-modified protein or functional protein fragment.

In certain embodiments, the protein or functional protein fragment immobilized on the surface retains about 100 percent of the activity of the tetrazine-modified protein or functional protein fragment.

In certain embodiments of the above methods, the tetrazine-modified protein or tetrazine-modified functional protein fragment is an enzyme or functional fragment thereof, a binding protein or functional fragment thereof, or an antibody or functional fragment thereof.

In certain embodiments of the above methods, the surface is a glass surface, a metal surface, a polymer surface, or a bead surface.

In certain embodiments of the above methods related to selective orientation of immobilized protein, retention of protein activity on immobilization, and quantitative protein loading on immobilization, the tetrazine-modified protein or tetrazine-modified functional protein fragment is prepared by genetic encoding using a non-canonical amino acid bearing a tetrazine moiety represented by the formula (II):

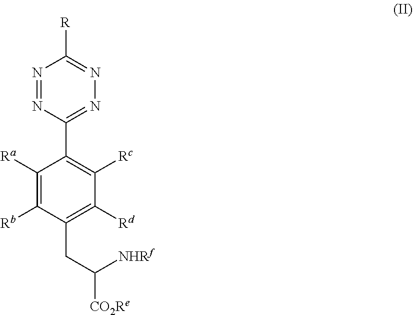

or a stereoisomer or salt thereof, wherein

R is selected from substituted or unsubstituted C1-C6 alkyl group and substituted or unsubstituted phenyl group;

R^{a}, R^{b}, R^{c}, and R^{d} are independently selected from hydrogen, C1-C3 alkyl, C1-C3 haloalkyl, C1-C3 alkoxy, and halo;

R^{e} is hydrogen, a counter ion, or a carboxyl protecting group; and

R^{f} is hydrogen or an amine protecting group.

In certain embodiments of these methods, R is selected from substituted or unsubstituted C1-C6 alkyl group; R^{a}, R^{b}, R^{c}, and R^{d} are hydrogen; R^{e} is hydrogen, a counter ion, or a carboxyl protecting group; and R^{f} is hydrogen or an amine protecting group.

In certain embodiments of these methods, the tetrazine-modified protein or tetrazine-modified functional protein fragment is prepared by genetic encoding using a non-canonical amino acid bearing a tetrazine moiety such as 4-(6-methyl-s-tetrazin-3-yl)phenylalanine (Tet-v2.0-methyl), 4-(6-ethyl-s-tetrazin-3-yl)phenylalanine (Tet-v2.0-ethyl), 4-(6-isopropyl-s-tetrazin-3-yl)phenylalanine (Tet-v2.0-isopropyl), or 4-(6-butyl-s-tetrazin-3-yl)phenylalanine (Tet-v2.0-n-butyl).

In other embodiments of the above methods related to selective orientation of immobilized protein, retention of protein activity on immobilization, and quantitative protein loading on immobilization, the tetrazine-modified protein or tetrazine-modified functional protein fragment is prepared by genetic encoding using a non-canonical amino acid bearing a tetrazine moiety represented by the formula (I):

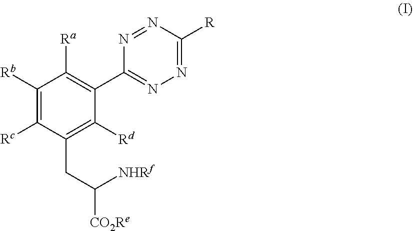

or a stereoisomer or salt thereof, wherein

R is selected from substituted or unsubstituted C1-C6 alkyl group and substituted or unsubstituted phenyl group;

R^{a}, R^{b}, R^{c}, and R^{d} are independently selected from hydrogen, C1-C3 alkyl, C1-C3 haloalkyl, C1-C3 alkoxy, and halo;

R^{e} is hydrogen, a counter ion, or a carboxyl protecting group; and

R^{f} is hydrogen or an amine protecting group.

In certain embodiments of these methods, R is selected from substituted or unsubstituted C1-C6 alkyl group; R^{a}, R^{b}, R^{c}, and R^{d} are hydrogen; R^{e} is hydrogen, a counter ion, or a carboxyl protecting group; and R^{f} is hydrogen or an amine protecting group.

In certain embodiments of these methods, wherein the tetrazine-modified protein or functional protein fragment is prepared by genetic encoding using a non-canonical amino acid bearing a tetrazine moiety such as 3-(6-methyl-s-tetrazin-3-yl)phenylalanine (Tet-v3.0-methyl), 3-(6-ethyl-s-tetrazin-3-yl)phenylalanine (Tet-v3.0-ethyl), 3-(6-isopropyl-s-tetrazin-3-yl)phenylalanine (Tet-v3.0-isopropyl), 3-(6-t-butyl-s-tetrazin-3-yl)phenylalanine (Tet-v3.0-t-butyl), or 3-(6-n-butyl-s-tetrazin-3-yl)phenylalanine (Tet-v3.0-n-butyl).

For the compounds described herein, in certain embodiments, R^{a}, R^{b}, R^{c}, and R^{d} are hydrogen, in other embodiments, R^{a}, R^{b}, and R^{d} are hydrogen, and in further embodiments, R^{a} and R^{d} are hydrogen.

In certain embodiments, the compounds of the invention are amino acids and maybe exist in neutral (e.g., —NH₂ and —CO₂H) or ionic form (e.g., —NH₃⁺ and —CO₂⁻) depending on the pH of the environment. It will be appreciated that the compounds of the invention include a chiral carbon center and that the compounds of the invention can take the form of a single stereoisomer (e.g., L or D isomer) or a mixture of stereoisomers (e.g., a racemic mixture or other mixture). It will be appreciated that the individual stereoisomers and mixtures of isomers are useful in methods of the invention for incorporating tetrazine-containing residues into proteins and polypeptides.

The preparation of representative tetrazine non-canonical amino acids, methods for genetic encoding proteins and polypeptides using the tetrazine non-canonical amino acids, and proteins and polypeptides comprising the tetrazine non-canonical amino acids is described in WO 2016/176689 (PCT/US2016/030469), expressly incorporated herein by reference in its entirety.

The tetrazine non-canonical amino acids of formulae (I) and (II) are useful for genetic encoding proteins to provide the tetrazine-modified protein or tetrazine-modified functional protein fragment and their subsequent use in the methods of the invention to provide the surfaces of the invention. It will be appreciated that the tetrazine non-canonical amino acids of formula (I) have certain advantages over the tetrazine non-canonical amino acids of formula (II). For example, it is important to note that the amino-acyl tRNA synthetase enzyme that recognizes the compounds of formula (I) (i.e., Tet3.0 compounds) are functional in both prokaryotic and eukaryotic systems, which makes them more versatile non-canonical amino acids relative to the compounds of formula (II) (i.e., Tet2.0 compounds) (Tet2.0 compounds only work in E. coli).

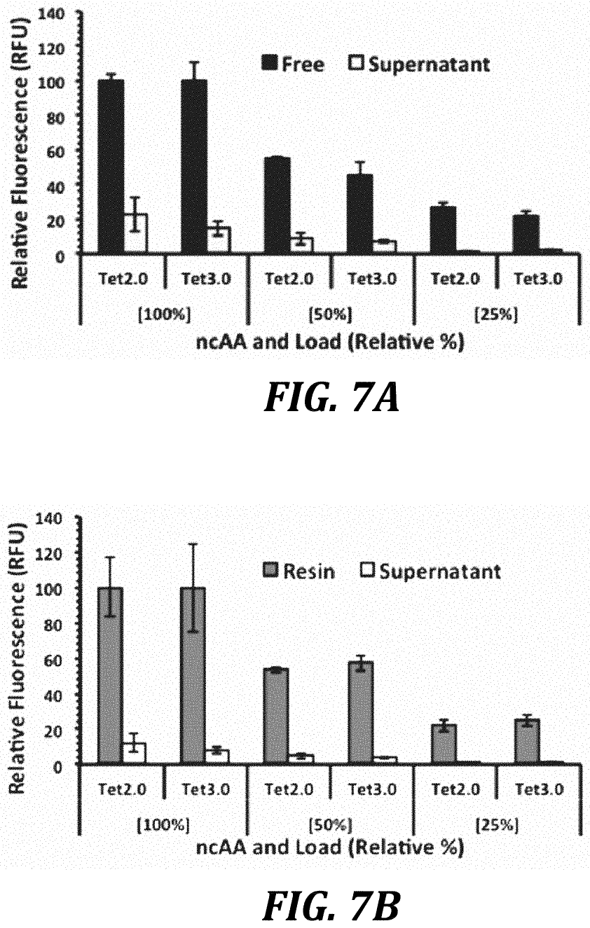

A comparison of the effectiveness of surface immobilization with representative Tet3.0 compounds (e.g., compounds of formula (I)) compared to representative Tet2.0 compounds (e.g., compounds of formula (II)) is described below and shown in FIGS. 7A and 7B.

In another aspect, the invention provides a surface having a protein or functional protein fragment immobilized thereon obtainable by the methods of the invention.

In certain embodiments, the surface having a protein or functional protein fragment immobilized thereon comprises a protein or functional protein fragment covalently coupled a surface, wherein the protein or functional protein fragment is a tetrazine-modified protein or a tetrazine-modified functional protein fragment, and wherein the tetrazine-modified protein or the tetrazine-modified functional protein fragment has been genetically encoded to include a tetrazine moiety at a predetermined amino acid site, wherein the surface is a trans-cyclooctene-modified surface, and wherein the protein or functional protein fragment is covalently coupled to the surface via the reaction of the tetrazine of the tetrazine-modified a protein or functional protein fragment with the trans-cyclooctene of the trans-cyclooctene-modified surface.

In these embodiments, the tetrazine-modified protein or the tetrazine-modified functional protein fragment is prepared by genetic encoding using a non-canonical amino acid bearing a tetrazine moiety as described herein (e.g., a compound of formulae (I) or (II)).

In a further aspect, the invention provides a method for measuring the binding of a ligand to a protein or functional protein fragment. In certain embodiments, the method comprises contacting a ligand with a surface of the invention as described herein; and determining whether the ligand binds to the protein or functional protein fragment immobilized on the surface. In the above method, measuring the binding of a ligand to a protein or functional protein fragment can be used as a screening process useful in therapeutic drug discovery.

The following is a description of representative embodiments of the invention.

The majority of genetically encodable bioorthogonal reactions available for protein immobilization proceed at sluggish reaction rates or require conditions that compromise protein functionality. For example, the commonly used strain-promoted azide-alkyne click reaction proceeds at a rate of ˜0.1-10 M⁻¹ s⁻¹ which necessitates high protein concentrations and overnight incubations that tend to promote non-specific protein adsorption, protein aggregation and loss in enzyme activity. The use of a copper catalyst can increase the azide-alkyne click reaction rate by 10-100 fold but this still results in a reaction half-life of about 3 hrs at 1 μM concentration of both reagents. Worse yet, the reactive oxygen species generated by copper-catalyst azide-alkyne click reactions have been well documented to affect the structure and functional integrity of proteins, making it impossible to predict the quantity of functional protein immobilized with this approach.

In contrast, the inverse-electron demand Diels-Alder reaction between tetrazines and strained trans-cyclooctenes (sTCOs) is one of the fastest bioorthogonal reactions in existence and represents an ideal candidate for such a protein-limited immobilization reaction. The reaction tolerates a range of buffered conditions appropriate for protein handling, requires no catalyst, and boasts tunable rate constants of up to 10⁶ M⁻¹ s⁻¹. The slower reacting TCO functionality can also be incorporated site-specifically into proteins in the form of a cyclooctene-lysine and has been successfully used in cells for protein labeling. Unfortunately, this TCO amino acid is prone to isomerization into its unreactive cis form, limiting its use in quantitative applications. Alternatively, site-specifically incorporation of a tetrazine-containing (Tet2.0) non-canonical amino acid (ncAA) into proteins has been reported, and the second-order in proteo reaction rate have been observed for this ncAA with sTCO is upwards of ˜72,000 M⁻¹ s⁻¹—high enough to allow rapid, quantitative, and sub-stoichiometric labeling of proteins in live cells, even at low concentrations.

The present invention utilizes the tetrazine-sTCO reaction (see FIG. 1A) to achieve protein-limited immobilization of two different proteins on beads and flat surfaces without the reaction compromising enzyme activity. By genetically encoding the Tet2.0 ncAA the site of incorporation within the protein can be controlled, and therefore the resulting orientation of the protein upon immobilization (FIG. 1B). Using genetically encoded Tet2.0 represents a rapid, gentle, and generalizable bioorthogonal immobilization reaction that permits control over protein load during immobilization on sTCO-functionalized surfaces with minimal effects on protein activity.

As described herein, a thermostable variant of human carbonic anhydrase II (tsCA) was selected as a “hard” enzyme (i.e. one characterized to undergo minimal structural changes upon interacting with surfaces) that is also a genuine candidate for constructing protein materials. tsCA is a zinc metalloenzyme with a small, rapidly diffusing substrate which has long been targeted for creating biomaterials (e.g., for carbon sequestration and artificial lungs. Tet2.0 was site-specifically incorporated Tet2.0 into tsCA in good yields using GCE at positions 186 (tsCA₁₈₆), 233 (tsCA₂₃₃), and 20 (tsCA₂₀) which would respectively orient the tsCA active-site towards the bulk solvent, parallel to, and towards the surface upon reaction with an sTCO-surface (see FIG. 1C).

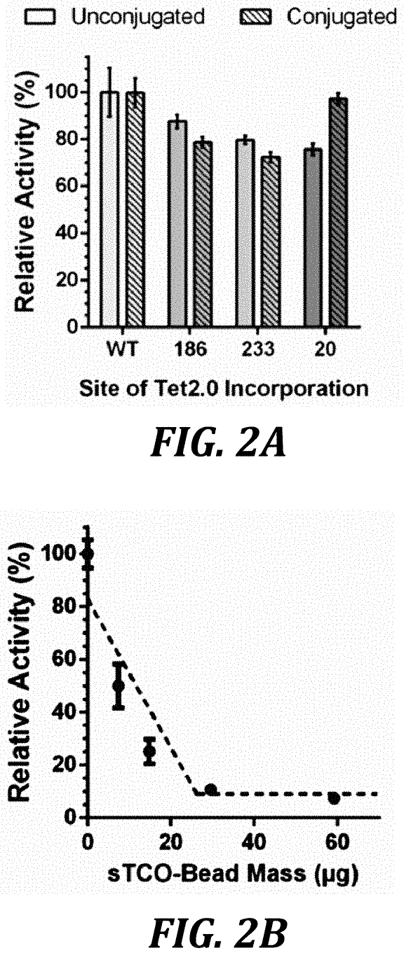

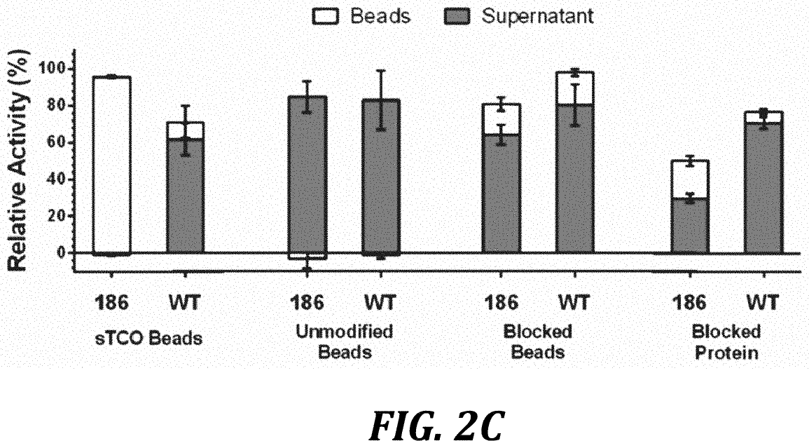

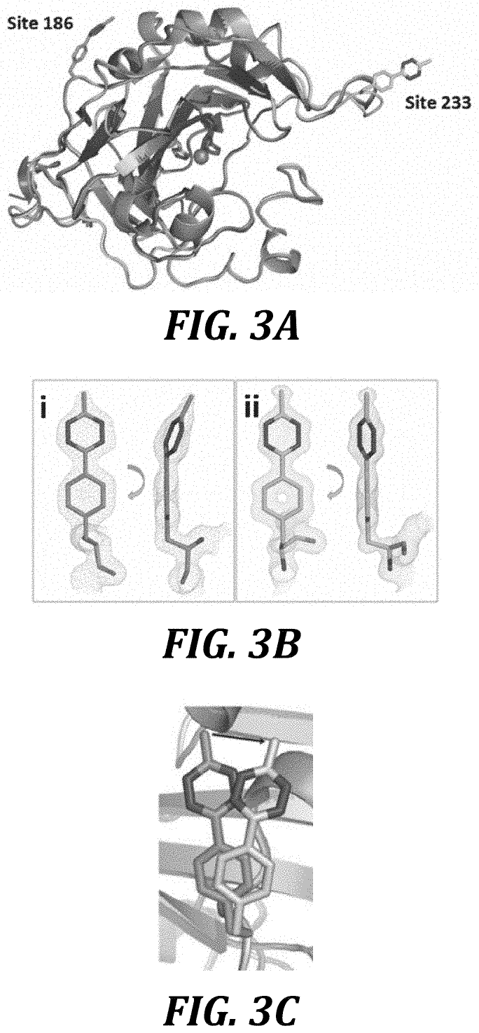

In order to define the effects of site-specific ncAA incorporation and the impacts of the Tet2.0 reaction conditions independently of protein immobilization, the extent to which tsCA's esterase activity is affected (i) by Tet2.0 incorporation at each site and (ii) by the subsequent reaction of Tet2.0 at these three sites with a large, soluble polymer “surface mimetic,” sTCO-PEG₅₀₀₀, were assessed. Tet2.0 incorporation at positions distant from the active-site (186 and 233) led to a slight about 10% (±3%) decrease in activity, whereas incorporation adjacent to the active-site at site 20 only led to a about 25% (±5%) decrease in activity. Subsequent conjugation with sTCO-PEG₅₀₀₀ led to a further (about 10%) decrease in activity for tsCA₁₈₆ and tsCA₂₃₃, but an increase in activity for tsCA₂₀, offsetting the change observed in the unconjugated protein (FIG. 2A). To discover any structural changes that may account for these activity changes, the structural characterizations of tetrazine ncAAs in proteins using X-ray crystallography were carried out. Crystal structures of tsCA₁₈₆ and tsCA₂₃₃ before and after reaction with sTCO-OH showed little change (C_{α}-RMSD≤0.2 Å), with the largest being a slight shift in the loop between residues 232-238 that may accommodate the tetrazine side-chain when incorporated at site 233 during crystal packing (FIG. 3A). Clear density for the Tet2.0 side-chains, which confirmed site-specific incorporation was observed. Interestingly, the tsCA₁₈₆ tetrazine ring showed deviations from the expected planarity (FIG. 3B), suspected to be due to radiation-induced reduction such as is known to occur during synchrotron data collection. Electron density corresponding to the ligated sTCO moiety upon either pre-reaction or in crystallo reaction of tsCA₁₈₆ or tsCA₂₃₃, was not observed indicating that this group has flexibility and is disordered in the crystal (FIG. 3C). Indirect evidences that the modification reaction occurred include crystal stress cracking upon soaking with sTCO and an about 6% change in the a-axis of the unit cell, as well as a 3.5 Å shift in the terminal methyl group of Tet2.0 at position 186 upon reaction with sTCO, presumably a side-chain movement to accommodate the ligation product (FIG. 3C). While no structure was determined for tsCA₂₀, tsCA₂₀ showed larger changes in activity upon Tet2.0 incorporation and polymer attachment given the site's close proximity to the active site. The lower activity of tsCA₂₀ followed by its regain in activity upon conjugation could be due to the relatively hydrophobic Tet2.0 side-chain nestling into the active-site pocket to impede substrate access, and its subsequent conjugation with sTCO-PEG₅₀₀₀ sterically hindering that packing and re-opening the active-site to allow substrate access.

tsCA₁₈₆ was used to develop a standard protocol for immobilizing tsCA on sTCO-modified magnetic beads, hypothesizing its activity would be least impacted by surface immobilization (FIG. 1C). The protocol includes a quick five-minute reaction at room temperature in a physiological buffer (either HEPES or phosphate at pH 7.5 at room temperature) followed by six washes that minimizes non-specific adsorption and has an associated about 15% loss of beads. A simple titration performed by exposing a fixed amount of protein to decreasing amounts of beads defined the bead binding capacity (FIG. 2B). To ensure that protein would always be limiting in our immobilizations, the maximal amount of protein applied in subsequent experiments (i.e., “100%” load) was set to be approximately half of the bead binding capacity.

To determine the specificity of the immobilization, the immobilization efficiency for tsCA₁₈₆ and tsCA_{WT} was compared under four conditions: exposure to (i) sTCO-beads; (ii) unmodified beads; (iii) sTCO-beads blocked with Tet2.0; and (iv) protein blocked with sTCO-OH exposed to sTCO-beads (FIG. 2C). For tsCA₁₈₆ exposed to sTCO-beads (condition i) only about 1% of the enzyme activity remained in supernatant and 95% of the activity was associated with the beads (FIG. 2C). The remaining conditions represent controls that should not effectively lead to protein immobilization, and as such, none of these control conditions led to a notable enrichment of enzyme activity associated with the beads. For all but one control condition (tsCA₁₈₆ under condition iv, addressed below), 60-80% of the enzyme activity remained in the supernatant while 0-20% of the activity was associated with the beads (FIG. 2C). The activity associated with the beads under these conditions reflects irreversible non-specific adsorption (i.e., not removed by washes). These trends can be rationalized as follows: non-specific adsorption is minimal for the unmodified beads (condition ii); however, hydrophobic surface modifications such as sTCO and sTCO-Tet2.0 (conditions i and iii) increase non-specific adsorption to about 10% to about 15%, respectively (FIG. 2C). Modification of tsCA₁₈₆ with a hydrophobic sTCO-OH (condition iv) increases non-specific adsorption further (FIG. 2C). In this instance, some of this non-specific adsorption is likely reversible in nature and is removed during washing, which accounts for the low (about 50%) overall recovery of protein activity, with only 20% of this being associated with the beads. It is also possible that under this condition, the loss in total recovered activity may be due to structural changes that occur to the enzyme as a result of the interaction with the beads thereby reducing the activity of the residual enzyme left in the supernatant. Taken together, these experiments show that the Tet2.0-sTCO reaction is performing as intended—with virtually 100% immobilization occurring, and at most 10% of this being non-specific in nature, as determined by the non-specific adsorption of tsCA_{WT} to sTCO-modified beads (FIG. 2C). Moreover, because at least 90% of the protein is specifically immobilized by the action of the Tet2.0-sTCO reaction, and the Tet2.0 is site-specifically incorporated into the protein, it is concluded that at least 90% of the protein present must also be site-specifically oriented.

Given these positive results based on tracking enzyme activity, protein-limited immobilization was validated with tracking the mass balance of all protein in all fractions using the “gold standard” method of radioactivity. For generating metabolically ³⁵S-radiolabeled protein, tsCA₂₃₃—the highest expressing variant was used. tsCA₂₃₃ behavior like tsCA₁₈₆ with minimal tsCA₂₃₃ remaining in the supernatant was confirmed, and the majority retained on the sTCO-beads. These experiments also confirmed that enzyme activity and radioactivity track well with each other and importantly, that mass balance was maintained throughout the immobilization process.

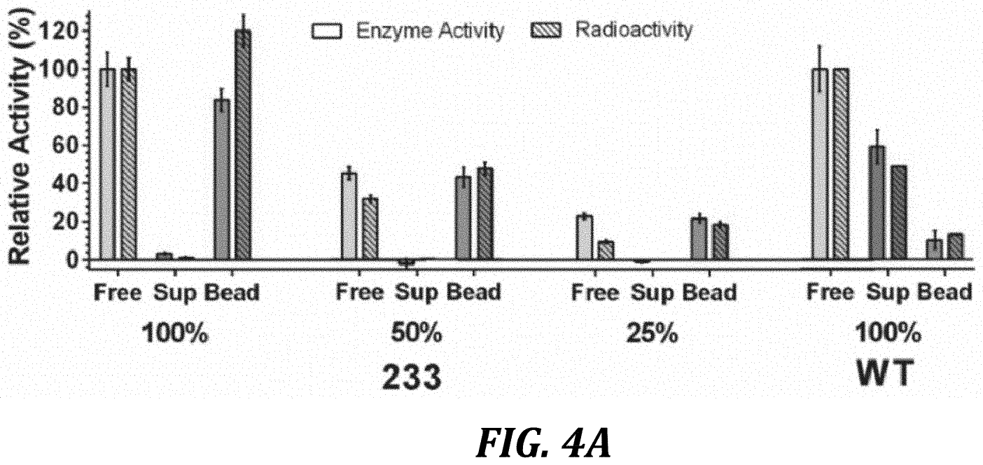

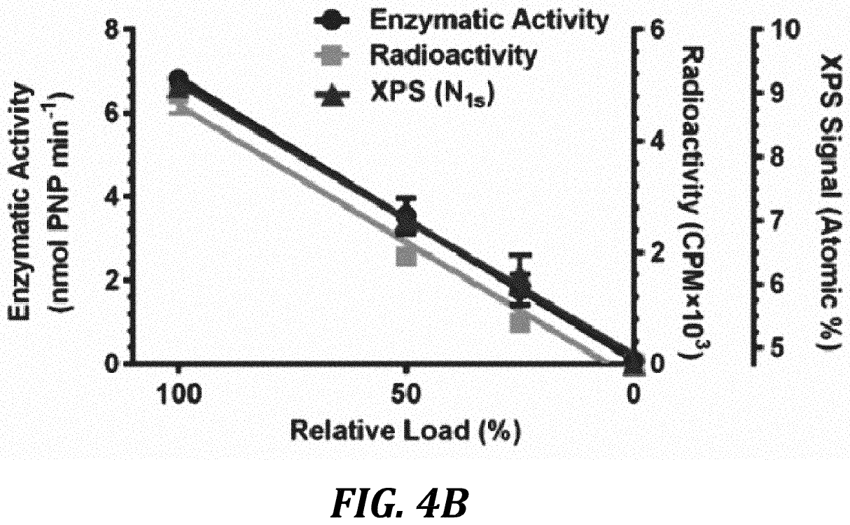

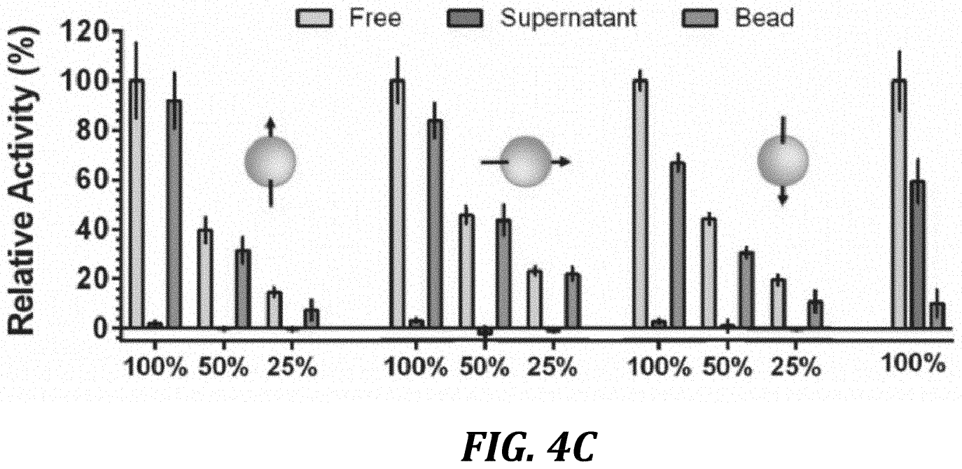

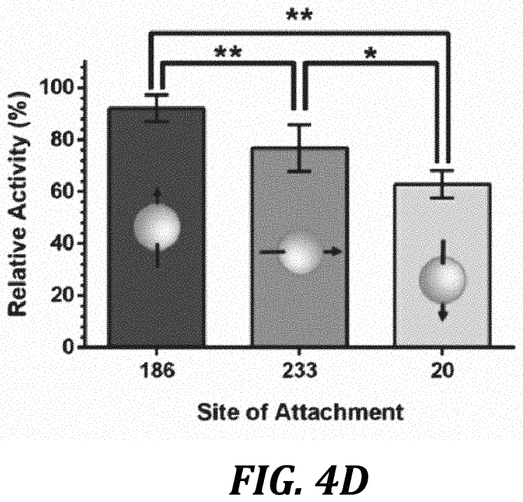

To demonstrate that the Tet2.0-sTCO reaction allows pre-specification of the amount of protein immobilized, tsCA₂₃₃ was applied to sTCO-beads at 100%, 50%, and 25% loads. At all three loads, both enzyme activity and radioactivity were completely depleted from supernatant and correspondingly associated with the beads (FIG. 4A). Furthermore, direct on-bead quantification by X-ray photoelectron spectroscopy (XPS)—a surface-sensitive characterization technique that permits quantification of the atomic percentages of the elements in proteins at the material surface—corroborates these observations. The atomic percentage of nitrogen directly observed on the beads increases in a load-dependent manner and closely trends with both enzyme activity and radioactivity (FIG. 4B). In all loads, radioactivity appeared to be slightly greater than enzymatic activity, which suggests that the enzymatic activity loss results from orientation interactions with the surface upon immobilization (discussed below). From these results, it was concluded that not only is depletion of supernatant activity an acceptable indicator of mass transfer to the beads in this instance, but that when using the Tet2.0-sTCO reaction with tsCA₂₃₃, the amount of protein loaded can be pre-specified with at least 90% being site-specifically immobilized.

Whether this ability extends to additional sites within tsCA was assessed by attempting equivalent differential loading experiments with tsCA₁₈₆ and tsCA₂₀. Similar to tsCA₂₃₃, a complete depletion of enzyme activity and corresponding increase in activity associated with the beads at all loads was observed for both variants (FIG. 4C). This confirms the ability to pre-specify the amount of oriented protein loaded via protein-limited immobilization is not limited to a single site, which allows for meaningfully correlation of orientation with activity. Comparing the activity at 100% load relative to their free-in-solution controls, immobilized tsCA₁₈₆, tsCA₂₃₃, and tsCA₂₀ have significantly different activities of 90%, 75%, and 60%, respectively when immobilized (FIG. 4D). Interestingly, there is also an indication that the activity of the three tsCA_{Tet2.0} orientations are also differentially sensitive to protein surface-density. Whether the changes in activity between these various orientations is solely a result of orientation (i.e. substrate accessibility) or is influenced by other variables such as site-specific structural changes arising in the protein upon immobilization that affect the dynamical/mechanistic steps in catalysis, or is an effect of the nanostructure of the surface itself, is difficult to assess without determination of the kinetic properties of tsCA_{Tet2.0} at each orientation (i.e. k_{cat} and Kₘ). These results highlight the types of novel, valuable information that can be derived when the immobilization reaction does not affect activity and protein load and orientation can both be precisely controlled.

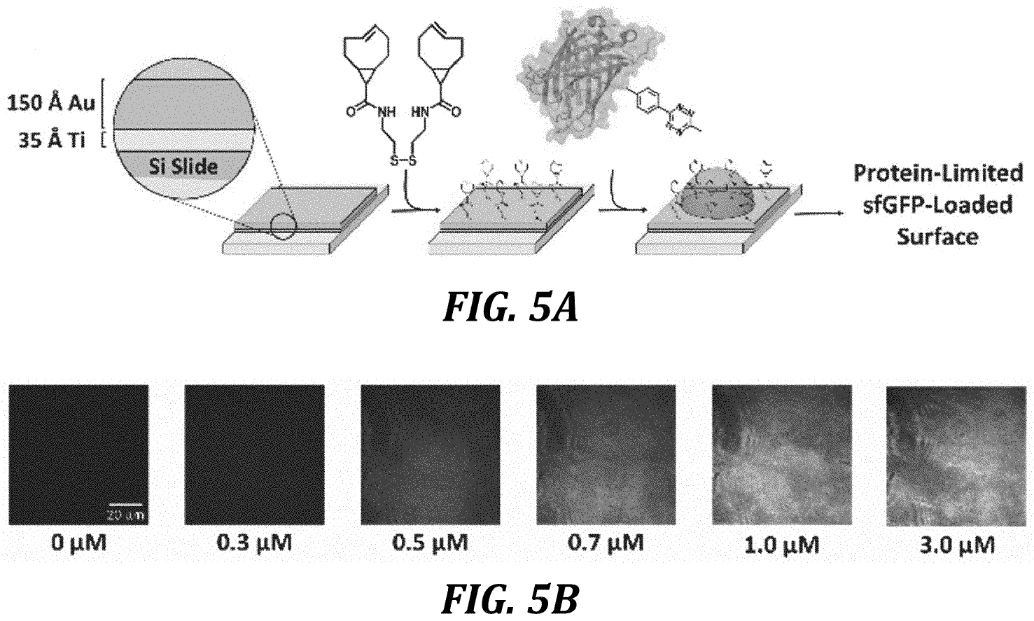

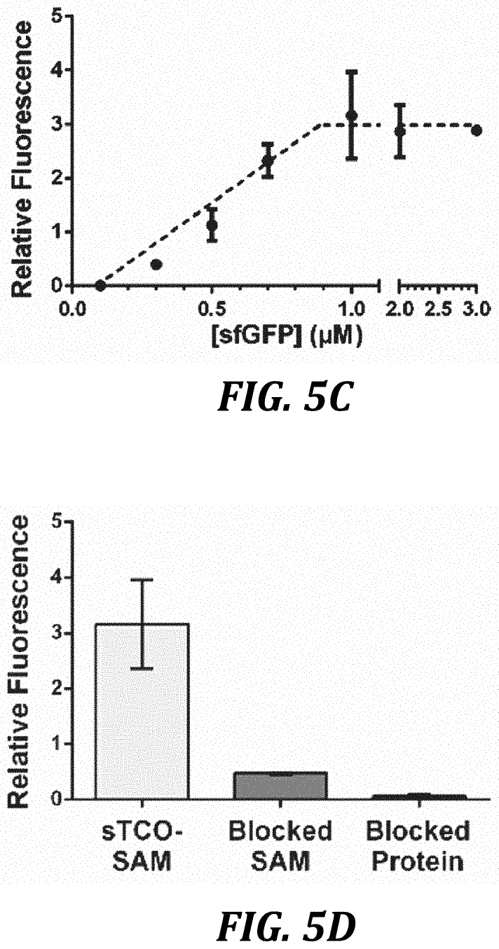

Because many applications of protein-based biomaterials and surface analysis techniques are optimized for flat surfaces, protein-limited immobilization on flat surfaces using sTCO-functionalized self-assembled monolayers (sTCO-SAMs) on gold formed from a di-sTCO disulfide reagent with a short linker that we synthesized (see FIG. 5A). For these experiments, superfolder green fluorescent protein was immobilized with Tet2.0 incorporated at site 150 (sfGFP₁₅₀) and quantify the amount of protein present using total internal reflection fluorescence (TIRF) microscopy, because even single molecules of sfGFP can be detected. To simultaneously determine the surface binding capacity and the ability to carry out protein-limited immobilization, the concentration of sfGFP₁₅₀ applied to the surface was varied, and noted that the surface-associated fluorescence increased linearly and then plateaued above approximately 1 μM sfGFP₁₅₀ (FIGS. 5B and 5C). To verify that the Tet2.0-sTCO reaction was responsible for immobilization, blocking experiments similar to those performed for tsCA on sTCO-beads were carried out (FIG. 5D). Similarly, it was observed that pre-reacting either the surface with Tet-PEG₅₀₀₀, or sfGFP₁₅₀ with sTCO-PEG₅₀₀₀ abolished the ability to immobilize protein, indicating that both a reactive Tet2.0 and sTCO are required for effective immobilization. These results confirm that we are achieving orientation-controlled protein-limited immobilization of sfGFP₁₅₀ onto sTCO-SAMs, and thereby demonstrate the generalizability of this approach to multiple surface morphologies and proteins.

As described herein, the speed, bioorthogonality, and mild conditions of the reaction between genetically-encoded Tet2.0 and sTCO provides access to protein-limited immobilization and provides a generalizable approach to precisely control the amount and orientation of immobilized proteins. The ability to control load by modulating the amount of protein introduced to the system at low protein concentration (nM) with short reaction times (minutes) minimizes protein denaturation and non-specific adsorption, allowing for protein-loaded surfaces to be prepared with exceptional levels of homogeneity. Moreover, a site within tsCA has been identified that experiences minimal activity losses upon immobilization and/or polymer attachment. Due to this minimal loss of activity and the ease with which tsCA can be produced and enzymatically detected, these Tet2.0-tsCA constructs can serve as useful loading controls for work with more challenging enzymes.

With this expanded ability for precise construction of protein-based biomaterials, it becomes possible to meaningfully define how variables such as protein quantity, orientation, and density impact protein function/stability and interplay with surface-dependent variables such as roughness and nanostructure, and to then take advantage of those relationships to create improved materials. For example, precise control over orientation will permit activity density to be maximized (e.g. FIG. 1D) and thereby facilitate further miniaturization in protein-based biosensors.

### Abbreviations

GCE genetic code expansion

PEG polyethylene glycol

ncAA non-canonical amino acid

sTCO strained-trans-cyclooctene

tsCA thermostable carbonic anhydrase

HEPES 4-(2-hydroxyethyl)-1piperazineethanesolfonic acid

XPS X-ray photoelectron spectroscopy;

sfGFP superfolder green fluorescent protein

SAM self-assembled monolayers

TIRF total internal reflection fluorescence

As used herein, the term “about” refers to ±1 percent of the specified value.

The following examples are provided for the purpose of illustrating, not limiting the invention.

Materials and Methods

All solutions were prepared using ultrapure (Type 1) water which was deionized using a Synergy UV water purification system equipped with a Biopak Polisher (MilliporeSigma, USA) to a resistivity of ≥18 MΩ.

Purchased chemicals were used without further purification. Anhydrous dichloromethane was prepared by overnight stirring with calcium hydride and distillation under argon atmosphere. All sTCO-derivatives were stored either as a dried powder or as a solution at −20° C. away from light. Tet2.0 was stored at room temperature in the powdered form. Thin-layer chromatography (TLC) was performed on silica 60E-254 plates (MilliporeSigma, USA). The TLC spots of alkenes were charred by potassium permanganate staining. Flash chromatographic purifications of synthetic products were performed using a CombiFlash Rf MPLC system with silica gel 60 columns (230-400 mesh size) (Teledyne ISCO, USA). ¹H NMR spectra were recorded on Bruker 400 MHz and 700 MHz instruments and ¹³C NMR spectra were recorded at 175 MHz. Chemical shifts are shown in ppm with the residual non-deuterated solvent peaks of CDCl₃ (δ=7.26 in ¹H NMR, δ=77.23 in ¹³C NMR), CD₃OD (δ=3.31 in¹H NMR, δ=49.2 in ¹³C NMR), or d₆-DMSO (δ=2.5 in ¹H NMR, δ=39.5 in ¹³C NMR) serving as internal standards. Splitting patterns of protons are designated as singlet (s), doublet (d), triplet (t), quartet (q), multiplet (m), doublet of doublets (dd). Mass spectrometry spectra are from a Waters Synapt G2 mass spectrometer coupled to a 2D nanoAcquity liquid chromatography system (Waters Corporation, USA).

Compound Synthesis

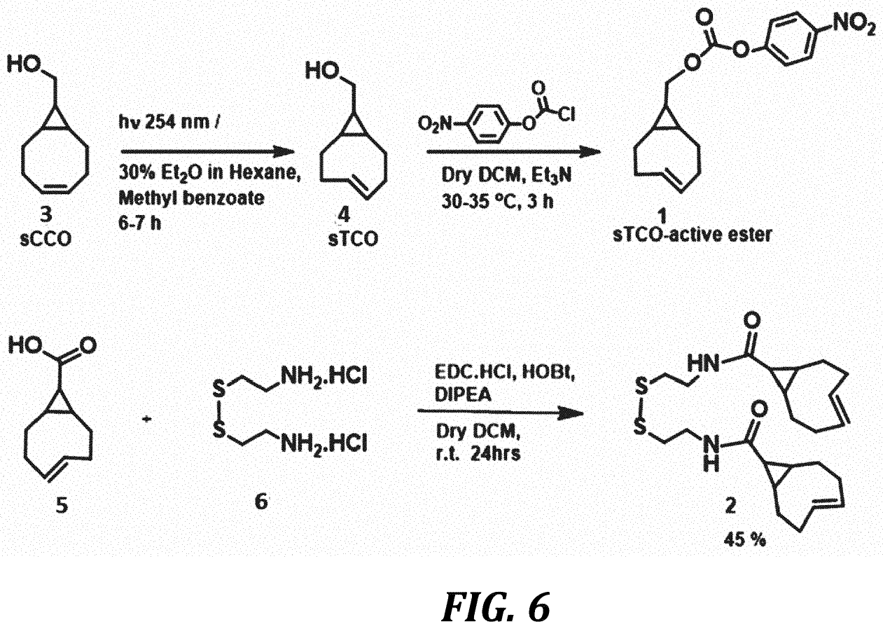

A schematic illustration of the synthesis of 4-nitrophenyl active ester of trans-cyclooctene (sTCO) and disulfide attached di-sTCO derivatives is shown in FIG. 6.

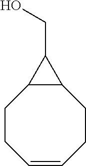 [SMILES: OCC1C2CC/C=C\CCC12]

(Z)-Bicyclo[6.1.0]non-4-en-9-ylmethanol (sCCO, 3): Prepared as described in O'Brien, J. G. K.; Chintala, S. R.; Fox, J. M. Stereoselective Synthesis of Bicyclo[6.1.0]Nonene Precursors of the Bioorthogonal Reagents S-Tco and Bcn. The Journal of Organic Chemistry 2018, 83 (14), 7500-7503, DOI: 10.1021/acs.joc.7b02329. ¹H NMR (700 MHz, CD₃OD) δ 5.64-5.60 (2H, m), 3.38 (2H, d, J=7 Hz), 2.30-2.26 (2H, m), 2.19-2.15 (2H, m), 2.09-2.04 (2H, m), 1.73-1.43 (2H, m), 0.79-0.74 (2H, m), 0.58-0.55 (1H, m).

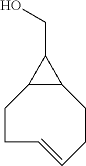 [SMILES: OCC1C2CC/C=C/CCC12]

(E)-Bicyclo[6.1.0]non-4-en-9-ylmethanol (sTCO, 4): Prepared as described in Royzen, M.; Yap, G. P. A.; Fox, J. M. A Photochemical Synthesis of Functionalized Trans-Cyclooctenes Driven by Metal Complexation. Journal of the American Chemical Society 2008, 130 (12), 3760-3761, DOI: 10.1021/ja8001919. ¹H NMR (700 MHz, CD₃OD) δ 5.89-5.84 (1H, m), 5.15-5.10 (1H, m), 3.44-3.39 (2H, m), 2.37 (1H, d, J=13.3 Hz), 2.27 (1H, dt, J=12.6, 4.2 Hz), 2.25-2.23 (1H, m), 2.19-2.15 (1H, m), 1.94-1.87 (2H, m), 0.92-0.87 (1H, m), 0.64-0.58 (1H, m), 0.50-0.46 (1H, m), 0.37-0.31 (2H, m).

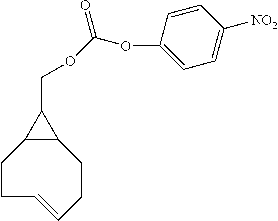 [SMILES: O=C(OCC1C2CC/C=C/CCC21)Oc1ccc([N+](=O)[O-])cc1]

(E)-bicyclo[6.1.0]non-4-en-9-ylmethyl (4-nitrophenyl) carbonate, 1): In a dry round-bottom flask, sTCO 4 (0.3 gm, 1.97 mmol) was dissolved in anhydrous dichloromethane (DCM) under inert atmosphere. Subsequently, trimethylamine (Et₃N) (650 μL, 4.9 mmol) and 4-nitrophenyl chloroformate (0.43 gm, 2.16 mmol) were added to the solution and stirred at 30-35° C. for 2-3 hrs. After consumption of all starting material (monitored by TLC), 15 mL of DCM was added to the reaction mixture and washed with water. The aqueous layer was re-extracted twice with DCM. The organic layers were combined, dried with anhydrous Na₂SO₄, and concentrated using rotary evaporator. Purification was performed using silica gel flash column chromatography (5% ethyl acetate in hexane) yielded yellowish white solid material 1 (0.51 gm, 1.6 mmol). Yield 81%. ¹H NMR (400 MHz, CDCl₃) δ 8.27 (2H, d, J=9.6 Hz), 7.37 (2H, d, J=9.6 Hz), 5.88-5.82 (1H, m), 5.18-5.14 (1H, m), 4.18 (2H, d, J=7.2 Hz), 2.43-2.39 (1H, m), 2.35-2.22 (3H, m), 1.96-1.90 (2H, m), 0.94-0.83 (1H, m), 0.69-0.64 (1H, m), 0.62-0.49 (3H, m).

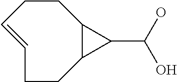 [SMILES: OC(O)C1C2CC/C=C/CCC21]

(E)-Bicyclo[6.1.0]non-4-ene-9-carboxylic acid (sTCO-CO2H, 5): In a dry quartz flask, the cis-isomer of sTCO-CO₂H (33, 34) (1.2 g, 7.22 mmol) and methyl benzoate (2.2 mL, 18.06 mmol) were dissolved in 400 mL of solvent (hexane:ether=1:1). The flask was placed in a Rayonet reactor and connected via PTFE tubing to a column and FMI pump. The column was packed with dry silica (60 Å, 6 cm) and silver impregnated silica (17 gm). The column was rinsed with ether and set to a circulation flow rate of 80 mL/min and 16 low pressure mercury lamps (2537 Å) turned were applied during this circulation. The photolysis of the reaction mixture continued for 7 hr. The column was washed with an additional 400 mL of ether then silica was poured into a 500 mL Erlenmeyer flask. The silica was stirred with saturated aq. sodium chloride solution (200 mL) and methylene chloride (200 mL) for 15 min. After filtration of silica, the aqueous fraction was extracted with dichloromethane (3 times). The combined organic layers were again washed with 100 ml water, dried with anhy. Na₂SO₄, and concentrated under reduced pressure to afford the trans isomer of the bi-cyclooctene derivative 5. ¹H NMR (400 MHz, CDCl₃) δ 5.91-5.83 (1H, m), 5.20-5.12 (1H, m), 2.40 (1H, d, J=4.8 Hz), 2.32-2.22 (3H, m), 2.03-1.92 (2H, m), 1.37-1.33 (1H, m), 1.28-1.21 (1H, m), 0.96-0.89 (2H, m), 0.65 (1H, q, J=12.8 Hz). ¹³C NMR (175 MHz, CD₃OD) δ 178.8, 139.1, 132.7, 39.1, 34.4, 33.1, 28.5, 28.3, 28.3, 27.4.

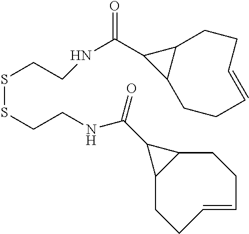 [SMILES: O=C(NCCSSCCNC(=O)C1C2CC/C=C/CCC21)C1C2CC/C=C/CCC21]

(4E,4′E)-N,N′-(Disulfanediylbis(ethane-2,1-diyl))bis(bicyclo[6.1.0]non-4-ene-9-carboxamide, 2): In a mixture of solvents comprising anhydrous dichloromethane (DCM) and N,N-dimethylformamide (DMF) (3:1, 5 mL), sTCO-CO₂H 5 (250 mg, 1.5 mmol), 1-ethyl-3-(3-dimethylaminopropyl) carbodiimide (EDC.HCl) (345 mg, 1.8 mmol) and hydroxybenzotriazole (HOBt) (243 mg, 1.8 mmol) were added under argon atmosphere and stirred for 30 minutes under ice cold conditions. Next, cystamine dihydrochloride 6 (191 mg, 0.75 mmol) was added, followed by the addition of N,N-diisopropylethylamine (DIPEA) (650 μl, 2.5 mmol) to the reaction mixture. After 15 minutes the ice bath was removed and stirring was continued for another 20 hours at room temperature. Then, 20 mL DCM added to the reaction mixture and washed with water. The aqueous layer was re-extracted twice with DCM. The organic layers were combined, washed with brine solution, dried with anhydrous Na₂SO₄, and concentrated using a rotary evaporator. Purification was done using silica gel flash column chromatography (10-15% methanol in dichloromethane) which yielded a white solid material 2 (143 mg, 0.316 mmol). Yield 42%. ¹H NMR (700 MHz, CDCl₃) δ 6.52 (2H, t, J=6.3 Hz), 5.91-5.86 (2H, m), 5.17-5.12 (2H, m), 3.56 (4H, q, J=6.3 Hz), 2.80 (4H, t, J=6.3 Hz), 2.38 (2H, d, J=13.3 Hz), 2.30-2.22 (6H, m), 1.99-1.91 (4H, m), 1.25-1.22 (2H, m), 1.13-1.09 (2H, m), 0.92-0.87 (2H, m), 0.80 (2H, t, J=4.2 Hz), 0.61 (2H, q, J=11.9 Hz). ¹³C NMR (175 MHz, CDCl₃) δ 174.2, 138.4, 131.6, 38.7, 38.3, 38.2, 33.6, 33.2, 29.4, 27.5, 25.8, 24.6. ESI-MS calcd for C₂₄H₃₇N₂O₂S₂ ([M+H]⁺) 449.2291. found 449.2299.

Chloride salt of (S)-2-amino-3-(4-(6-methyl-1,2,4,5-tetrazin-3-yl)phenyl) propanoic acid (7, Tet2.0): Synthesis was carried out using a modified synthetic round, as described previously (Popchock, A. R.; Jana, S.; Mehl, R. A.; Qiu, W. Engineering Heterodimeric Kinesins through Genetic Incorporation of Noncanonical Amino Acids. ACS Chemical Biology 2018, 13 (8), 2229-2236, DOI: 10.1021/acschembio.8b00399) to further increase yields as follows. In a flame dried 50 mL heavy walled reaction tube Boc-protected 4-CN phenylalanine (500 mg, 1.72 mmol) was combined with Ni(OTf)₂ (306 mg, 0.86 mmol) and acetonitrile (0.9 ml, 17.2 mmol) under argon atmosphere. Next anhydrous hydrazine (2.7 mL, 86 mmol) was slowly added to the reaction mixture and purged with argon for 5 to 10 minutes and the reaction vessel immediately sealed, and the reaction mixture then heated to 50° C. for 24 hr. Following, the reaction mixture was cooled to room temperature, opened slowly and 20 eqv. of 2 M NaNO₂ in a 5 mL water solution was added. Next, the reaction mixture was washed with ethyl acetate (1×20 ml) to remove the homo coupling product. The collected aqueous phase was acidified with 4 M HCl (pH about 2) under ice cold conditions and extracted with ethyl acetate (3×30 mL). The combined organic layers were washed with brine, dried with anhydrous Na₂SO₄ and concentrated under reduced pressure. Silica gel flash column chromatography purification (30-35% ethyl acetate in hexanes with 1% acetic acid) provide 485 mg of Boc-protected Tet2.0 (1.35 mmol, 78%) in the form of a pinkish red gummy material. ¹H NMR (400 MHz, CDCl₃) δ 8.54 (2H, d, J=7.6 Hz), 7.44 (2H, d, J=8.0 Hz), 5.06 (1H, d, J=6.0 Hz), 4.71 (bs, 1H), 3.37-3.19 (2H, m), 3.1 (s, 3H), 1.44 (s, 9H). ¹³C NMR (175 MHz, CDCl₃) δ 175.3, 167.2, 163.9, 155.3, 141.2, 130.5, 130.4, 128.1, 80.4, 54.1, 37.9, 28.3, 21.2.

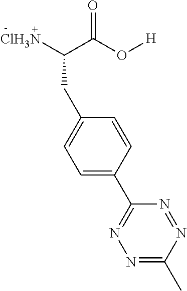

The purified Boc-protected Tet2.0 amino acid (450 mg, 2.23 mmol) was dissolved in 5 mL ethyl acetate and charged with 3 mL HCl gas saturated 1,4-dioxane under argon atmosphere. The reaction mixture was allowed to stir at room temperature until the starting materials was consumed, as monitored by TLC (typically 3 to 4 h). The resulting product was concentrated under reduced pressure and re-dissolved in ethyl acetate (2×10 mL) and similarly concentrated to remove excess HCl gas which resulted in pink colored solid material of Tet2.0 7 in quantitative yield (98%). ¹H NMR (700 MHz, CD₃OD) δ 8.56 (2H, d, J=7.0 Hz), 7.60 (2H, d, J=7.7 Hz), 4.39 (1H, bs), 3.48-3.33 (2H, dd, J=3.5, 14 Hz)), 3.1 (s, 3H). ¹³C NMR (175 MHz, CD₃OD) δ 171.2, 169.1, 165.3, 140.7, 133.3, 131.7, 129.6, 55.1, 37.4, 21.3. ESI-MS calculated for C₁₂H₁₄N₅O₂ ([M+H]⁺) 260.114. found 260.1.

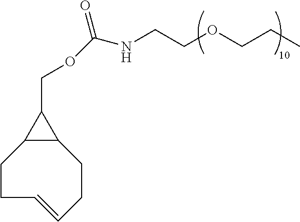

mPEG5000-linked sTCO (sTCO-PEG5000, 8): In 3 mL of anhydrous dichloromethane, 65 mg (0.013 mmol) of mPEG5000-Amine (Laysan Bio, Inc., USA) were dissolved. The activated ester of sTCO 1 (6 mg, 0.16 mmol) was subsequently added, followed by triethylamine (10 μL, 0.05 mmol) both of which were added under argon atmosphere. The reaction mixture was stirred at room temperature for 24 hours. After that, the solvent was concentrated onto silica gel under reduced pressure and purified to yield the desired compound 8 (47 mg, 0.009 mmol) by silica gel column chromatography (5% methanol in dichloromethane). Yield 69%. ¹H NMR (400 MHz, CD₃OD) δ 5.90-5.82 (1H, m), 5.17-5.09 (1H, m), 3.91 (2H, d, J=6.8 Hz), 3.82-3.79 (3H, m), 3.63 (411H, bs), 3.54-3.50 (4H, m), 3.45 (2H, t, J=4.8 Hz), 3.35 (3H, s), 3.26 (2H, t, J=5.6 Hz), 3.16 (2H, q, J=7.6 Hz), 2.35 (1H, d, J=15.2 Hz), 2.27-2.15 (3H, m), 1.97-1.88 (2H, m), 0.95-0.84 (1H, m), 0.66-0.54 (2H, m), 0.48-0.41 (2H, m).

Molecular Cloning

The tsCA thermostable variant is described in a 2006 Canadian Patent No. 2541986 (also U.S. Pat. No. 7,521,217). The variant contains six mutations (A65T, L100H, K154N, L224S, L240P, and A248T) discovered via random mutagenesis to individually increase thermostability. The codon-optimized gene for tsCA expression in E. coli was ordered from ATUM (formerly DNA 2.0) in a pJ201 plasmid. The plasmid was digested using NcoI and XhoI restriction enzymes (Thermo Scientific, USA) and ligated into a pBad expression vector. Amber stop codons (TAG sites) were introduced into tsCA by conventional overlap extension PCR using the primers described in Table 1 (Bryksin, A. V.; Matsumura, I. Overlap Extension Pcr Cloning: A Simple and Reliable Way to Create Recombinant Plasmids. BioTechniques 2010, 48 (6), 463-465, DOI: 10.2144/000113418).

TABLE 1
Primers, plasmids, and strains used.
Primers

| Name | Sequence | Length |
|---|---|---|
| pBAD tsCA | TTTTTGGGCTAACAGGAGGAATTAACCATGGCGCATCATTGGGGTT | 46 |
| Fwd | SEQ ID NO: 1 | |
| pBAD tsCA | TCTTCTCTCATCCGCCAAAACAGCCAAGCTTTTAGTGATGGTGGTG | 46 |
| Rev | SEQ ID NO: 2 | |
| tsCA- | GTCTGCTGCCGTAGAGCCTGGATTAC | 26 |
| TAG186 For | SEQ ID NO: 3 | |
| tsCA- | GTAATCCAGGCTCTACGGCAGCAGAC | 26 |
| TAG186 Rev | SEQ ID NO: 4 | |
| tsCA- | GAATTTCAATGGTTAGGGCGAGCCGGAAG | 29 |
| TAG233 For | SEQ ID NO: 5 | |
| tsCA- | CTTCCGGCTCGCCCTAACCATTGAAATTC | 29 |
| TAG233 Rev | SEQ ID NO: 6 | |
| tsCA-TAG20 | GGCACAAAGATTAGCCAATTGCGAAG | 26 |
| For | SEQ ID NO: 7 | |
| tsCA-TAG20 | CTTCGCAATTGGCTAATCTTTGTGCC | 26 |
| Rev | SEQ ID NO: 8 | |

Plasmids

| | | | Size | Addgene | |
|---|---|---|---|---|---|
| Name | Promoter | Resistance | (bp) | ID | Notes |
| pBAD-tsCA- | araBAD | Ampicillin | 4757 | 105665 | Houses a copy of tsCA_{WT} |
| WT | | | | | |
| pBAD-tsCA- | araBAD | Ampicillin | 4757 | 105837 | Houses a copy of tsCA₁₈₆ with a |
| TAG186 | | | | | TAG site at position 186 |
| pBAD-tsCA- | araBAD | Ampicillin | 4757 | 105838 | Houses a copy of tsCA₁₈₆ with a |
| TAG233 | | | | | TAG site at position 233 |
| pBAD-tsCA- | araBAD | Ampicillin | 4757 | 105666 | Houses a copy of tsCA₁₈₆ with a |
| TAG20 | | | | | TAG site at position 20 |
| pBAD- | araBAD | Ampicillin | 4829 | 85482 | Houses a copy of sfGFP_{WT} |
| sfGFP-WT | | | | | |
| pBAD- | araBAD | Ampicillin | 4829 | 85483 | Houses a copy of sfGFP₁₅₀ with a |
| sfGFP- | | | | | TAG site at position 150 |
| 150TAG | | | | | |
| pDule- | lpp (aaRS) | Tetracycline | 6333 | 85496 | Houses a suppression pair |
| Tet2.0 | glnS | | | | including a Tet2.0-specific |
| | (tRNA_{CUA}) | | | | MjTyrRS and a cognate, recoded |
| | | | | | tRNA_{CUA} |

Strains

| Number | Plasmid 1 | Plasmid 2 | Resistance | Notes |
|---|---|---|---|---|
| 1 | pBAD-CA-WT | N/A | Ampicillin | Used for production of tsCA_{WT} |
| 2 | pBAD-CA- | pDule-Tet2.0 | Ampicillin/ | Used for production of tsCA₁₈₆ |
| | TAG186 | | Tetracycline | |
| 3 | pBAD-CA- | pDule-Tet2.0 | Ampicillin/ | Used for production of tsCA₂₃₃ |
| | TAG233 | | Tetracycline | |
| 4 | pBAD-CA- | pDule-Tet2.0 | Ampicillin/ | Used for production of tsCA₂₀ |
| | TAG20 | | Tetracycline | |
| 5 | pBAD-sfGFP- | N/A | Ampicillin | Used for production of sfGFP_{WT} |
| | WT | | | |
| 6 | pBAD-sfGFP- | pDule1- | Ampicillin/ | Used for production of sfGFP₁₅₀ |
| | 150TAG | Tet2.0 | Tetracycline | |

The construction of pBAD-sfGFP plasmids and pDule1-Tet2.0 were carried out as previously described (Plass, T.; Milles, S.; Koehler, C.; Szymanski, J.; Mueller, R.; Wießler, M.; Schultz, C.; Lemke, E. A. Amino Acids for Diels-Alder Reactions in Living Cells. Angewandte Chemie International Edition 2012, 51 (17), 4166-4170, DOI: 10.1002/anie.201108231; Stokes, A. L.; Miyake-Stoner, S. J.; Peeler, J. C.; Nguyen, D. P.; Hammer, R. P.; Mehl, R. A. Enhancing the Utility of Unnatural Amino Acid synthetases by Manipulating Broad Substrate Specificity. Molecular BioSystems 2009, 5 (9), 1032-1038, DOI: 10.1039/B904032C). It should be noted here that all plasmids used here were deposited to and are available from Addgene (see Table 1 for Addgene ID number).

To prepare the strains outlined in Table 1, chemically competent E. coli DH10B cells were chemically transformed by incubation of approximately 25 μg each of a plasmid housing the gene of interest and a machinery plasmid housing the suppression machinery (orthogonal amino-acyl tRNA synthetase enzyme specific for Tet2.0 and a cognate orthogonal suppressor tRNA_{CUA}; see Table 1) if necessary, as specified in Table 1, for about 15 min on ice prior to incubation at 42° C. for 45 s. Cells were then recovered in 1 mL of SOC media (10 mM MgSO₄ and 0.4% glucose in 2×YT) by shaking at 37° C. for about 1 h before being plated on LB agar plates with the appropriate antibiotics. Individual colonies were selected and grown in 5 mL of 2×YT under appropriate antibiotic selection overnight prior to being frozen in 15% glycerol and stored at −80° C. until needed. Overnight starter cultures grown at 37° C. in 5 mL of 2×YT under proper antibiotic selection were used to inoculate larger expression cultures.

Protein Expression, Amber Suppression, and Purification

50 mL cultures were inoculated from overnight starter cultures under proper antibiotic selection in non-inducing media (Studier, F. W. Stable Expression Clones and Auto-Induction for Protein Production in E. Coli. Methods Mol Biol 2014, 1091, 17-32, DOI: 10.1007/978-1-62703-691-7-2) supplemented with 200 μM ZnSO₄ and 500 μM Tet2.0 (diluted from a 100 mM solution in DMF). Cells were grown for about 48 h at 37° C. with constant shaking at 250 rpm in an 126 incubator-shaker (Eppendorf, KGaA, Germany; formerly New Brunswick Scientific, USA) in capped plastic 500 mL baffled flasks. They were harvested by centrifugation at 5,500 rcf for 10 min and the pellets stored at −80° C. To purify protein, cell pellets were thawed on ice and resuspended in 5 mL of TALON Wash buffer (50 mM NaH₂PO₄, 500 mM NaCl, 5 mM Imidazole, pH 7.0) and subsequently microfluidized at 18,000 psi using a M-110P microfluidizer (Microfluidics Corp., USA). To remove insoluble cell debris, microfluidized lysate was centrifuged at about 21,000 rcf for 30 min at 4° C. No more than 50 mL of cleared lysate was then incubated with 1.0 mL of washed TALON Cobalt resin (Takara Bio, Japan) for about 1 h at 4° C. with frequent agitation. The TALON resin was then transferred to a 10 mL column and washed with 50 mL of TALON Wash buffer. To elute, 3.0 mL of TALON Elution buffer (50 mM NaH₂PO₄, 500 mM NaCl, 250 mM imidazole, pH 7.0) was added to the column, the first 0.5 mL being discarded as dead volume. The resulting 2.5 mL of eluate was transferred to a PD-10 de-salting column (GE Healthcare) and de-salted according to manufacturer's instructions into HEPES Buffer consisting of 100 mM HEPES (pH 7.5), 150 mM NaCl and 1 μM ZnSO₄. After de-salting, purified protein was spin-concentrated using a 15 mL Vivaspin-2 10 kDa MWCO disposable spin-concentrator (GE Healthcare, USA) according to manufacturer's instructions. Unless otherwise stated, protein concentration was determined by A₂₈₀ measurement on a NanoDrop 2000 spectrophotometer using the following molar extinction coefficients: tsCA_{WT}, 50070 M⁻¹ cm⁻¹; tsCA₂₀, tsCA₁₈₆, and tsCA₂₃₃, 61724 M⁻¹ cm⁻¹; sfGFP_{WT}, 24080 M⁻¹ cm⁻¹; sfGFP₁₅₀, 35734 M⁻¹ cm⁻¹. Purified protein samples were stored for several hours at 4° C. before being aliquoted and flash frozen by liquid nitrogen and stored at −80° C. until needed. They were not re-frozen after thawing and kept no longer than 7 d at 4° C.

sfGFP was purified similarly, with notable modifications being a 24 hr expression in autoinduction media not supplemented with ZnSO₄, and protein being desalted into “PBS Buffer” consisting of 50 mM Na₂PO₄, 100 mM NaCl, pH 7.0.

Protein Mass Spectrometry

Purified protein was diluted to a concentration of approximately 50 μM in either HEPES Buffer (tsCA) or PBS Buffer (sfGFP) and desalted using C₄ ZipTips (MilliporeSigma, USA). Desalted protein was eluted from the C₄ ZipTip using 50:50 MQ water:acetonitrile containing 0.01% formic acid and analyzed using electrospray ionization on a LTQ FT Ultra High Performance Mass Spectrometer (Thermo Scientific, USA) at Oregon State University's Mass Spectrometry Facility and deconvoluted using a multiple overlapping peak maximum entropy deconvolution software (SpectrumSqaure, USA).

tsCA Bioconjugation and Size Exclusion Chromatography

tsCA variants were diluted to 2 mg/mL (about 67 μM) and combined with 10 equivalents of sTCO-PEG₅₀₀₀ (about 667 μM) and allowed to react for approximately 5 min at room temperature in HEPES Buffer. Reacted tsCA was then run through a Superdex S200 10/300 size exclusion column in-line with an AKTA Explorer 100 FPLC (Amersham Biosciences, UK) at room temperature. In short, 1 mg of protein was loaded onto a column pre-equilibrated with HEPES Buffer and run isocratically at a rate of 0.3 mL/min, collecting 1.0 mL fractions and monitoring the absorbance at 280 nm. Protein purity was checked by SDS-PAGE.

Preparation of sTCO-Beads and tsCA Immobilization

sTCO-microparticles were prepared in 25 mg batches from commercially produced amine-functionalized magnetic microparticles. The manufacturer reports that these superparamagnetic microparticles (BioMag Amine magnetic microparticles, Bangs Laboratories Inc., USA) are composed of iron oxide with an amine-terminated overlayer comprised of a proprietary silane, are approximately 1.5 μm in diameter, and possess an overall irregular morphology while the surface roughness is not reported. 500 μL of 51 mg/mL BioMag Amine magnetic microparticles were washed three times with 1.0 mL of methanol, and three times again with 1.0 mL of dichloromethane to remove any residual surfactant. Washed beads were resuspended in 500 μL of anhydrous dichloromethane transferred to a 1.0 mL glass vial, combined with 10 mg of activated sTCO (compound 1) and 5.54 of DIPEA, backfilled with Ar gas, capped, parafilmed, coated with foil and left to react for approximately 24 hrs at room temperature with constant agitation. After the reaction had completed, sTCO-functionalized microparticles were washed three times with 1.0 mL of dichloromethane, and three times again with 1.0 mL of methanol before being resuspended in 1.0 mL of methanol and stored in a 1.0 mL sealed glass vial coated with foil at 4° C.

To immobilize protein, 59.2 μg of sTCO-microparticles were washed twice with MQ-H₂O and again 4 times with 100 mM HEPES buffer (pH 7.5, supplemented with 1 μM ZnSO₄) and resuspended in 25 μL of HEPES buffer per replicate. To this washed microparticle solution 254 of protein solution containing 0.125 nmol of protein (3.75 μg) were added to create a solution with a final bead concentration of 1.184 mg/mL and protein concentration of 2.5 μM. This amount of protein and beads corresponds with the typical 100% load; for the 50% and 25% load amounts, the bead amount was kept constant while the protein amount was reduced to 0.063 nmol (1.875 μg; final concentration of 1.125 μM) and 0.031 (937.5 ng; final concentration of 625 nM), respectively. This solution was allowed to react at room temperature for approximately 5 mins with frequent agitation. The microparticles were then separated from the supernatant by magnetic pull-down and washed three times with 1.0 mL of HEPES buffer supplemented with 0.05% Triton X-100 to remove non-specifically adsorbed protein, and three times again with 1.0 mL HEPES buffer before being resuspended in 500 μL of HEPES buffer. A 500 μL solution of beads afforded enough material to perform two activity assays, which were averaged as technical replicates. In a typical experiment, a 50 μL master solution of 5 μM tsCA is prepared per each replicate—this master solution was then split into two 25 μL aliquots, one of which was exposed to sTCO-microparticles as described above, whereas the remaining aliquot was diluted to 500 μL to serve as a free-enzyme control for activity assays. Likewise, the post-reaction supernatant was also collected and diluted to a final volume of 500 μL to accompany activity assays; when necessary, each 1.0 mL wash solution was collected and the activity of the resulting solution was mathematically multiplied by a factor of two, to correct for dilution. An approximate 15% loss in beads was observed during the washing steps (primarily through beads sticking to the walls of the polypropylene tubes in which samples were handled), to account for this, all bead-associated activity values were mathematically adjusted by multiplying by a factor of 1.176. When appropriate, the sTCO-beads were blocked with Tet2.0 via pre-reaction in a solution of 100 μM Tet2.0 in HEPES buffer for approximately five minutes followed by three washes in HEPES prior to exposure to tsCA₁₈₆. Likewise, when appropriate, a 10 μM solution of tsCA₁₈₆ was blocked with sTCO-OH via addition of an equal volume containing 10 molar equivalents of sTCO-OH for approximately five minutes prior to exposure to sTCO-beads.

To analyze sTCO-beads by XPS, the beads were mounted onto a silicon wafer support. To achieve this, silicon substrates were cut to 1×1 cm² (Oregon State University cleanroom), cleaned by sequential rinsing in H₂O (MilliQ Direct-Q3) and acetone (Pharmco-Aaper), and then sequentially sonicated in dichloromethane, acetone and ethanol (Pharmco-Aaper). Following sonication steps, 30 μl of functionalized Fe beads suspended in H₂O were added to the surface in 10 μl aliquots then the surfaces were dried under vacuum. The addition of Fe beads was done 3 times resulting in a uniform coating of functionalized Fe beads on the silicon substrates. The substrates were stored under nitrogen and away from light until analysis.

Preparation of sTCO-SAMs and sfGFP Immobilization

Silicon wafer substrates were cleaned by soaking in MilliQ water overnight followed by rinsing in MilliQ water and acetone the next morning. The silicon substrates were then cleaned by sonication in DCM (dichloromethane), acetone, and ethanol then dried under a stream of nitrogen and stored until thermal evaporation (VEECO Thermal Evaporator). Substrates were prepared by thermal evaporation of 3.5 nm of titanium (99.995%; Kurt. J. Lesker) followed by 100 nm of gold (99.999% pure; Kurt. J. Lesker) onto a clean silicon wafer. Gold-coated wafers were immersed in an ethanolic solution of sTCO-disulfide 2 or 1-dodecanthiol (Sigma Aldrich, 98% purity), a control for contact angle measurements, at a concentration of 1 μM. These solutions were prepared by either dissolving the reagent directly in ethanol, for the case of 1-dodecanthiol, or by diluting a 100 mM stock of the compound dissolved in DMF, in the case of 2. The samples were parafilmed and backfilled with nitrogen for 24 hours in the absence of any light. After the 24 hours the samples were thoroughly rinsed in ethanol and dried under nitrogen. Samples were then stored under nitrogen in the absence of light until needed, being stored no later than a week before being used for protein immobilization

Contact Angle Analysis of sTCO-SAMs

A contact angle goniometer (First Ten Angstroms, Portsmouth, Va.) was used to measure the contact angle of water on each substrate. Droplets (10 μL) were pipetted onto each surface and a high-resolution image was collected (n=3). The droplet shape, relative to the horizon line, was traced and a contact angle was generated by the provided instrument software.

sfGFP Immobilization

For protein immobilization, a 2 μL solution of sfGFP at varying concentrations was deposited onto the sTCO-SAM in triplicate and allowed to react for approximately 5 minutes at room temperature under ambient humidity. To quench the reaction, a 1 μL aqueous solution of 100 μM sTCO-PEG₅₀₀₀ was added to the 2 μL protein droplet and given approximately 10 minutes to completely react. The surface was subsequently washed under a stream of buffer (50 mM NaHPO₄, 100 mM NaCl, pH 7.50), followed by a stream of 50:50 buffer/deionized water, followed by a final stream of 100% deionized water for approximately 1 minute. When appropriate, sfGFP₁₅₀ was blocked with sTCO-PEG₅₀₀₀ by pre-reacting the protein at 1.0 μM with 10 molar equivalents of sTCO-PEG₅₀₀₀ for approximately five minutes in PBS buffer at room temperature prior to exposure to the sTCO-SAM surface. Likewise, when appropriate, the sTCO-SAM surface was blocked via pre-reaction with 1 uL of 100 Methyl-Tetrazine-mPEG₅₀₀₀ in water (Click Chemistry Tools, USA) at the location of eventual sfGFP₁₅₀ application for approximately five minutes at room temperature. Following immobilization, approximately 10 μL of buffer (50 mM NaHPO₄, 100 mM NaCl, pH 7.50) was then applied to the surface and a coverslip mounted, with moderate pressure applied to remove excessive buffer from between the coverslip and slide. The coverslip was then sealed to the slide using a store-bought nail polish preparation to prevent water loss during TIRFm observation.

Total Internal Reflection Fluorescence Microscopy (TIRFm) Analysis

To determine the relative amount of sfGFP present on the surface, fluorescent images of the surface were taken using an Axio Observer Z1 objective-type TIRF microscope (Zeiss) equipped with a 100×/1.46 numerical aperture oil-immersion objective and a back-thinned electron multiplier charge-coupled device camera (Photometrics) on TIR mode at a total magnification of 1000× (field of view being approximately 82 μm×82 μm) with an angle setting locked at 45°, and excitation using a 488 nm laser. To determine fluorescence intensity, three distinct (different fields of view) images were taken near the center of each spot where protein immobilization occurred, and their average intensity determined using Fiji (ImageJ) image analysis software. The intensity of these three images were averaged and the mean of the three spots were then averaged to arrive at a final value for fluorescence intensity (total of nine images across three spots), with error bars representing the standard deviation between the three triplicate spot averages.

Enzyme Activity Assays

Enzyme activity was determined using a modified PNPA assay, as developed by Verpoorte, Mehta, and Edsall (Verpoorte, J. A.; Mehta, S.; Edsall, J. T. Esterase Activities of Human Carbonic Anhydrases B and C. The Journal of Biological Chemistry 1967, 242 (18), 4221-4229). In this assay, tsCA samples were diluted to a concentration of 250 nM in a 1.7 mL polypropylene microcentrifuge tube. tsCA protein solutions (240 μL at 250 nM) were added to a single well of a glass-coated 96-well microplate and combined with 60 μL of a 20 mM para-nitrophenyl acetate (PNPA; Thermo Scientific, USA) solution dissolved in 1,2-dimethoxyethane to yield a 300 μL solution with a final protein concentration of 200 nM, and PNPA concentration of 4 mM and 1,2-dimethoxyethane concentration of 20%. The choice of 1,2-dimethoxyethane over acetone was made since there is evidence that acetone is slightly inhibitory to bovine carbonic anhydrase. For assaying activity of protein immobilized on beads, 240 uL of the resulting 500 uL solution yielded after adequate washing was treated similarly to free-in-solution assays to yield a solution of similar protein, PNPA, and 1,2-dimethoxyethan concentrations. The final solution was then added to a BioTek Synergy2 plate reader (BioTek Instruments, Inc., USA) immediately following PNPA addition and the absorbance at 348 nm (PNPA isosbestic point) was monitored every 18 seconds for 306 seconds with constant shaking between measurements. The enzyme activity was determined as the slope of the increasing absorbance at 384 nm (A348/s) and was mathematically blanked using the slope of a blank solution containing only HEPES solution, or, sTCO-microparticles for on-particle activity measurements. To calculate specific activity, A348 changes were converted to nmols using an empirically-derived molar extinction coefficient for para-nitrophenol under assay conditions of 3586 M⁻¹. Unless otherwise stated, all activity readings composed of three replicates, each being the average of two technical replicates.

Metabolic Radiolabeling with ³⁵S and Detection of Radiolabeled tsCA

To produce ³⁵S-radiolabeled tsCA_{WT} and tsCA₂₃₃, strains 1 and 3 (Table 1) were grown in 25 mL of autoinduction media as mentioned prior, with additional supplementation of EasyTag™ EXPRESS35S Protein Labeling Mix (Perkin Elmer, Inc, USA) at a final concentration of 0.48 mCi/mL. Cultures were grown for 24 hrs before harvest and purified as previously mentioned, with lysis being carried out using BugBuster® Protein Extraction Reagent (Merck Millipore, KGaA, Germany). Protein was stored at 4° C. until needed. Protein quantification was achieved via Bradford assay (Bradford, M. M. A Rapid and Sensitive Method for the Quantitation of Microgram Quantities of Protein Utilizing the Principle of Protein-Dye Binding. Analytical Biochemistry 1976, 72 (1), 248-254). Unless otherwise mentioned, detection of all radiolabeled proteins was carried out by forming a mixture of 1 mL HEPES solution containing radiolabeled protein (either free or immobilized) and 9 mL of Ultima-Flo M liquid scintillation cocktail (Perkin Elmer Inc, USA) in a 20 mL borosilicate scintillation vial and counting for 5 minutes under the S35 setting in either a Beckman LS6500 or Beckman LS6000 liquid scintillation counter (Beckman Coulter Inc, USA), with radioactivity being reported as raw CPM values. It should be noted here that radioactive tsCA was not assayed for its enzymatic activity. As such, enzymatic activity and radioactivity measurements were performed on two separate preparations of tsCA_{WT} and tsCA₂₃₃.

X-Ray Photoelectron Spectroscopy

The XPS data was collected with a PHI 5600 system (Physical Electronics, USA) using a monochromatic Al Kα X-ray source (hν=1486.6 eV, 300 W, 15 kV) and take-off angle of 45° (angle between the surface normal and the axis of the analyzer beam). Atomic compositions were calculated from C₁ₛ, N₁ₛ, O₁ₛ, Fe₂ₚ, Au_{4f}, and Si₂ₚ peak areas obtained from survey and high-resolution scans (analyzer pass energy=187.85 eV and 23.5 eV for survey and high-resolution scans, respectively). The spectra were collected at fresh spots on the sample (n=3) and were charge corrected to the C₁ₛ aliphatic carbon binding energy at 285.0 eV and a linear background was subtracted for all peak area quantifications except Fe, which used a Shirley background. Error bars in the reported data represent the standard deviation of the atomic percent average of the three spots. The peak areas were normalized by the sensitivity factors provided by the manufacturer and surface concentrations were calculated using CASA XPS (Casa Software Ltd).

The amount of protein on the surface of the Fe beads can be followed by the nitrogen atomic percent determined from the Nis signal. The nitrogen from just the protein layer can be calculated by examining the attenuation of the Fe₂ₚ signal from the core of the magnetic beads after protein is covalently attached to the surface by equation 1.

N_{Norm}=Nₚ−Nₛ(Feₚ/Feₛ) Eq. 1

where, Nₛ and Feₛ are the measured N and Fe atomic percent, respectively, from the Fe beads prior to either sTCO or covalent attachment of protein; Nₚ and Feₚ are the measured N and Fe atomic percent, respectively, from the Fe beads after addition of sTCO and covalent attachment of protein; and N_{Norm} is the nitrogen atomic percent that accounts for just the covalently attached protein.

Protein Crystallography

Expression, Purification, and Crystallization. Proteins were expressed in a similar fashion as described in Protein Expression, Amber Suppression, and Purification with the following modifications: culture volumes were 100 mL, and proteins were purified via a two-step process by first passing cleared lysate in TALON wash buffer over a HisTrap HP 5 mL column and eluting in TALON elution buffer followed by purification over a Superdex S200 10/300 column, both performed on an AKTA Explorer FPLC (columns and FPLC from Amersham Biosciences, UK). The proteins were de-salted into 10 mM HEPES (pH 7.5) and spin-concentrated to 11 mg/mL and 15 mg/mL, for tsCA₁₈₆, and tsCA₂₃₃, respectively, and stored at 4° C.

Two approaches were taken to prepare crystals of tsCA₁₈₆ and tsCA₂₃₃ reacted with sTCO-OH 4. For tsCA₁₈₆, the protein was first crystallized as described below, and subsequently reacted with 4 in crystallo by soaking crystals in a solution of 880 μM 4 (final methanol concentration about 10%) in artificial mother liquor with cryoprotectant for at least 1 min before being frozen in liquid nitrogen. These crystals showed visible cracking during the sTCO soak, suggestive of an in crystallo Tet2.0-sTCO reaction. For tsCA₂₃₃, freshly purified protein was reacted in solution through exposure to two equivalents of 4 (final concentration of 4 was approximately 82 μM, with 9.67% methanol in 1 mL of 10 mM HEPES, pH 7.5) for 15 min. The protein was de-salted using a PD-10 desalting column (GE Healthcare, USA), spin-concentrated to a final concentration of about 20 mg/mL using a VivaSpin-2 10 k MWCO disposable spin-concentrator (GE Healthcare, USA), and stored at 4° C.

In all cases, the enzymes were crystallized at 4° C. in hanging drops. tsCA₁₈₆ was crystallized using a reservoir solution of 0.2 M ammonium sulfate and 30% PEG 4000. tsCA₂₃₃ (Ordered Tet2.0) was crystallized using a reservoir solution of 0.2 M ammonium acetate, 0.1 M sodium acetate trihydrate pH 4.6, and 30% PEG₄₀₀₀. tsCA₂₃₃ (Disordered Tet2.0) and tsCA₂₃₃-sTCO were crystallized using a reservoir solution of 0.2 M sodium chloride, 0.1 M Tris pH 8.5, and 25% PEG 3350. Crystals grew in clusters of plates which were separated into individual crystals for data collection.

Data Collection. For diffraction data collection at −170° C., all crystals were passed through artificial mother liquor containing cryoprotectant (15% for all except 20% glycerol for tsCA₁₈₆) and then cryo-cooled by plunging into liquid nitrogen. Data were collected at beamline 5.0.2 with λ=1.0 Å (tsCA₁₈₆, tsCA₂₃₃ (Ordered Tet2.0), tsCA₂₃₃ (Disordered Tet2.0), tsCA₂₃₃-sTCO) and beamline 5.0.3 with λ=0.976 Å (tsCA₁₈₆+sTCO) at the Advanced Light Source (Berkley, Calif.). Data were collected for 360° at a detector distance of D=180 mm with Δφ=0.25° and 0.1 s exposure for tsCA₂₃₃ (Ordered Tet2.0), at D=210 mm with Δφ=0.25° and 0.1 s exposure for tsCA₂₃₃ (Disordered Tet2.0) and tsCA_{233-sTCO}, and at D=220 mm with Δφ=1° and 3 s exposure for tsCA₁₈₆+sTCO. For tsCA₁₈₆, data were collected from 2 crystals, each for 360° at D=200 mm with Δφ=1° and 2 s exposures.

Images were processed using XDS (Kabsch, W. Xds. Acta Crystallographica Section D 2010, 66 (2), 125-132, DOI: doi:10.1107/50907444909047337) or Mosflm (Battye, T. G.; Kontogiannis, L.; Johnson, O.; Powell, H. R.; Leslie, A. G. Imosflm: A New Graphical Interface for Diffraction-Image Processing with Mosflm. Acta crystallographica. Section D, Biological crystallography 2011, 67 (Pt 4), 271-281, DOI: 10.1107/s0907444910048675) and the CCP4 (The Ccp4 Suite: Programs for Protein Crystallography. Acta crystallographica. Section D, Biological crystallography 1994, 50 (Pt 5), 760-763, DOI: 10.1107/s0907444994003112) suite of programs. For tsCA₁₈₆, images showing substantial decay based on visual examination were excluded. For the first and second crystals, the first 185 and 131 images, respectively, were included. In all cases, a CC_{1/2} of about 0.2 was the resolution cutoff criterion (see Table 1 for corresponding resolutions) and a random 5% of reflections were marked for cross-validation.

Structure Determination and Refinement. In all cases, the structures were solved using molecular replacement with a structure of human carbonic anhydrase II (PDB code 1CA2 with 97% sequence identity) as the search model. All manual model building was done in Coot (Emsley, P.; Lohkamp, B.; Scott, W. G.; Cowtan, K. Features and Development of Coot. Acta Crystallographica. Section D, Biological Crystallography 2010, 66 (Pt 4), 486-501, DOI: 10.1107/s0907444910007493). The Tet2.0 coordinates and crystallographic information file was generated using phenix.elbow (Moriarty, N. W.; Grosse-Kunstleve, R. W.; Adams, P. D. Electronic Ligand Builder and Optimization Workbench (Elbow): A Tool for Ligand Coordinate and Restraint Generation. Acta Crystallographica. Section D, Biological Crystallography 2009, 65 (Pt 10), 1074-1080, DOI: 10.1107/S0907444909029436) with restraints further manually edited to allow Tet2.0 to fit into high resolution density. Refinements were carried out using Phenix (Adams, P. D.; Afonine, P. V.; Bunkoczi, G.; Chen, V. B.; Davis, I. W.; Echols, N.; Headd, J. J.; Hung, L. W.; Kapral, G. J.; Grosse-Kunstleve, R. W.; McCoy, A. J.; Moriarty, N. W.; Oeffner, R.; Read, R. J.; Richardson, D. C.; Richardson, J. S.; Terwilliger, T. C.; Zwart, P. H. Phenix: A Comprehensive Python-Based System for Macromolecular Structure Solution. Acta Crystallographica. Section D, Biological Crystallography 2010, 66 (Pt 2), 213-221, DOI: 10.1107/s0907444909052925) with TLS and riding hydrogens for tsCA₁₈₆, tsCA₂₃₃ (Disordered Tet2.0), tsCA₂₃₃-sTCO, and tsCA₁₈₆+sTCO and unrestrained individual anisotropic refinement (wu=0) for tsCA₂₃₃ (Ordered Tet2.0).

Accession Numbers. Coordinates and structure factors for tsCA₁₈₆, tsCA_{186-sTCO}, tsCA₂₃₃ (Ordered Tet2.0), tsCA₂₃₃ (Disordered Tet2.0), and tsCA_{233-sTCO} have been deposited in the Protein Data Bank with accession numbers 6NJ2, 6NJ6, 6NJ3, 6NJ5, and 6NJ4, respectively.

Statistical Methods

Experiments were carried out in triplicate and error bars represent the standard deviation of the individual measurements. Reported p-values are based on heteroskedastic, two-tailed t-tests performed using Microsoft Excel. For enzyme activity and radioactivity measurements, each value represents the mean of three measurements, with each measurement being the mean of two technical replicates. For XPS, an average and standard deviation were calculated (n=3) for each sample type.

Protein Characterization

Yield and Suppression Efficiency. Site-specific incorporation of Tet2.0 was accomplished through the use of the standard amber suppression approach (Chin, J. W. Expanding and Reprogramming the Genetic Code of Cells and Animals. Annual Review of Biochemistry 2014, 83, 379-408, DOI: 10.1146/annurev-biochem-060713-035737). This method relies on the use of an orthogonal amino acyl-tRNA synthetase (aaRS) and its cognate tRNA_{CUA} (together referred to as a suppression pair) that is used to genetically direct the incorporation of a noncanonical amino acid (ncAA) at the position of an amber stop codon. As described herein, Tet2.0 incorporation was achieved through use of a previously designed and characterized suppression pair consisting of: (1) an MjTyrRS that has been engineered specifically to recognize Tet2.0, and (2) its cognate, recoded MjtRNA_{CUA}^{Tyr} originating from Methanocaldococcus jannashii that recognizes amber stop codons. Both components of the Tet2.0 suppression pair are orthogonal in E. coli and produce protein with high efficiency and fidelity only when ncAA is supplemented to the media.

For tsCA, optimal suppression and yield could be achieved through growth and autoinduction in autoinduction media for 48 hrs at 500 μM Tet2.0. Under these conditions, yields of approximately 20-50 mg of high-purity protein per liter of culture were achieved with good suppression efficiencies with yields of the Tet2.0-containing proteins being comparable to that of WT protein. The high expression yield of tsCA required supplementation of the autoinduction media with 200 μM ZnSO₄ to achieve consistent enzymatic activity of the variants, which may be due to the Zn ion being a limiting cofactor during expression in the defined autoinduction media.

For sfGFP, optimal expression required 24 hrs of expression at similar conditions (ZnSO₄ supplementation was not necessary) which resulted in high yields (about 100-200 mg of high-purity protein per liter of culture) and a suppression efficiency of 46% for sfGFP₁₅₀.

Metabolic S35 Radiolabeling of tsCA. tsCA contains three sulfur-containing residues (M1, C205, M240), which allowed incorporation of S³⁵-containing methionine and cysteine through metabolic incorporation. Metabolic incorporation was used as opposed to the more commonly used I¹²⁵ modification, because there is evidence that this method can lead to erroneous protein quantification results due either to free I¹²⁵ or changes in sorption properties of iodinated proteins. By tracking the amount of radioactivity in the culture supernatant, cell lysis flow-through, and final wash (assumed to be representative of the amount in each of the five 10 mL washes) and correcting for dilution, roughly 32% of the radioactivity was estimated to be cellularly incorporated, and of that approximately 17% and 21% of the total radioactivity was incorporated in tsCA_{WT} and tsCA₂₃₃. The radioactivity (CPM) per pmol of tsCA for both tsCA_{WT} and tsCA₂₃₃ was determined to be approximately 48.3 and 31 CPM, respectively, indicating that a typical 100% load of protein (3.75 μg, or 125 pmol) should produce approximately 6000, and 3900 CPM for tsCA_{WT} and tsCA₂₃₃, respectively, which was observed to be empirically consistent and well above background.

Mass Spectrometry Analysis. Successful site-specific incorporation of Tet2.0 into tsCA was verified by electrospray ionization protein mass spectrometry. For tsCA, a tsCA_{WT} mass of 30002.2±1 Da was observed. tsCA_{WT} had a loss of 131.1±1 Da, and addition of 63.4±1 Da consistent with removal of N-terminal methionine and addition of Zn ion to the three active site histidine ligands, respectively. The incorporation of Tet2.0 at tsCA glutamate sites 186 and 233 showed the appropriate mass shift of 112.1 and 113.5±1 Da, respectively, while incorporation at phenylalanine site 20 showed the appropriate mass shift of 95.6 Da. Samples also showed +23.0±1 Da, corresponding to the mass of sodium adducts. No other peaks were observed that would correlate with background incorporation of natural amino acids. For sfGFP we observed similar results, with the sfGFP_{WT} variant matching the predicted mass, and sfGFP₁₅₀ showing a mass change consistent with Tet2.0 incorporation, 128.0±1 Da, (expected 127.3±1 Da). These samples also showed +23.0±1 Da, corresponding to the mass of sodium adducts. Taken together, these results corroborate SDS-PAGE analysis of purified tsCA and GFP protein confirm that Tet2.0 has been site-specifically incorporated into tsCA and sfGFP.

Enzymatic Activity. To assess enzymatic activity, an adapted version of a common esterase activity assay developed for carbonic anhydrase was employed (Altissimo, M.; Kiskinova, M.; Mincigrucci, R.; Vaccari, L.; Guarnaccia, C.; Masciovecchio, C. Perspective: A toolbox for protein structure determination in physiological environment through oriented, 2D ordered, site specific immobilization. Struct Dyn 2017, 4 (4), 044017, DOI: 10.1063/1.4981224). This assay relies on the innate esterase activity of carbonic anhydrase towards para-nitrophenol acetate (PNPA), which is a colorless compound that is hydrolyzed by tsCA to the colored product para-nitrophenol (PNP). To avoid any influences of pH that may arise from tsCA's interconversion of ambient CO₂ to HCO₃ prior to activity readings, colorimetric increases were monitored at PNP's isosbestic point (438 nm).

Overall, little effect of Tet2.0 incorporation on the enzymic activity was observed for tsCA₁₈₆ and tsCA₂₃₃ (about 10% decrease). For tsCA₂₀ on the other hand, a consistent about 25% reduction in activity was observed, the reasons for which may likely be due to its proximity to the active site. Subsequent reaction of Tet2.0 with an sTCO-PEG₅₀₀₀ polymer led to a further slight decrease in activity for tsCA₁₈₆ and tsCA₂₃₃ (about 10%), but conversely for tsCA₂₀ seemed to restore activity of this variant to that similar to tsCA_{WT}.

Tet2.0 Reactivity. To assess the in proteo reactivity of Tet2.0, an SDS-PAGE mobility shift assay and size exclusion chromatography (SEC) was relied on. Briefly, Tet2.0-containing protein is reacted with an excess (approximately 10 molar equivalents) of sTCO-PEG₅₀₀₀ polymer for approximately 5 minutes to allow complete reaction. This sample is then analyzed by either SDS-PAGE followed by Coomassie staining, or by SEC. If successful bioconjugation has occurred, a protein mobility shift is observed due to the attachment of a large polymer, which is detectable as an increase in the apparent molecular weight of the protein by SDS-PAGE or by decrease in retention volume by SEC. The ratio of unshifted (unreacted) and shifted (reacted) bands, as determined by densitometry analysis for SDS-PAGE analysis and by comparison of the reacted and reacted peaks for SEC, provides a means of assessing the in proteo reactivity of Tet2.0.

SDS-PAGE analysis revealed a nearly complete bioconjugation of protein (95-99% mobility shift) within 5 minutes at a protein concentration of approximately 9 μM for all three tsCA variants and sfGFP₁₅₀, confirming that these locations are accessible and sufficient for efficient immobilization within the 5-minute immobilization period. Importantly, no appreciable mobility shift for tsCA_{WT} or sfGFP_{WT} was observed, which suggests that the reaction between Tet2.0 and sTCO is specific and bioorthogonal. By SEC, both the reacted and unreacted proteins are effectively resolved, with a decrease in retention time of approximately 2 mL being observed for the reacted (retention volume of about 15.1 mL) protein relative to the unreacted protein (retention volume of 17.2 mL), which permitted isolation of the PEGylated tsCA_{Tet2.0} variants.

X-Ray Crystallography of tsCA-Tet2.0 and ligation product. There is little information available to determine how bioorthogonal handles on proteins effect protein structure. Structural characterization of tetrazines and their reaction within proteins to assess the effects of Tet2.0 incorporation and its reaction with sTCO have on the structure of the tsCA enzyme are described herein. These structures provide details of the Tet2.0 structure in a protein environment with resolutions ranging between 1.01 and 1.60 Å. Two different crystal forms compatible with tsCA₂₃₃ were identified—one in which the Tet2.0 at site 233 is part of the crystal packing interface, and therefore ordered and able to be modeled (tsCA₂₃₃ (Ordered Tet2.0); space group P2₁2₁2₁), and another in which the Tet2.0 at site 233 is solvent exposed and disordered and was not modeled (tsCA₂₃₃ (Disordered Tet2.0); space group C2221). However, following reaction with sTCO-OH 4 only crystals of the space group where the Tet2.0 was disordered (tsCA_{233-sTCO}; space group C222₁) were obtained, and consequently, the ligation product was also disordered and could not be modeled. The structures presented in FIGS. 3A and 3B represent those structures for which we obtained clear density for the Tet2.0 (tsCA₂₃₃ (Ordered Tet2.0), tsCA₁₈₆). In the context of tsCA₂₃₃, the analysis focused on the structure in which the Tet2.0 is ordered, has clear 2Fₒ-F_{c} density, and could be modeled. In both tsCA₁₈₆ and tsCA₂₃₃ (Ordered Tet2.0), Tet2.0 has clear density for the amino acid sidechain. The Tet2.0 conformation varies between crystal forms and chains in the asymmetric unit. The lack of density for Tet2.0 in tsCA₂₃₃ (Disordered Tet2.0) and tsCA_{233-sTCO}, where Tet2.0 is solvent exposed, provides evidence for mobility of the Tet2.0 side chain within this crystal form. Attempts to capture an atomic resolution snapshot of the Tet2.0-sTCO ligation did not provide sufficient electron density to model the ligation product; however, it is worthy to note that only crystals for tsCA₂₃₃ in the C222₁ space group where the Tet2.0 is disordered were obtained, suggesting that the P2₁2₁2₁ space group cannot accommodate the ligation product.

Characterization of Surfaces

Contact Angle Analysis of sTCO-SAMs. Equilibrium contact angle reflects the relative strength of molecular interactions at the solid liquid interface an provides information about the first few monolayers of a surface. Thiolated sTCO to two other surfaces of 1-dodecanethiol and bare gold were compared. Static contact angle goniometry, with 10 μL ultrapure water as the test fluid, was used to measure the angle between droplet and substrate, with low angles less than 90° correlating to hydrophilicity and angles greater than 90° correlating to hydrophobicity. Contact angles of 94°±1°, 103°±1°, and 66°±1° were measured for the bare gold, 1-dodecanthiol, sTCO SAM surfaces, respectively. These results for the bare gold surface indicate that the surface is hydrophobic, which suggests that there is likely a partial monolayer of carbon contamination on the surface since pristine clean gold surfaces are typically hydrophilic. Modifying the surface to a self-assembled monolayer (SAM) of 1-dodecanethiol yields a surface contact angle that is hydrophobic (103°±1°), which is the expected outcome for 1-dodecanethiol. For self-assembled monolayers of thiolated sTCO, on the other hand, the contact angle results in a hydrophilic surface. which may likely be due to the polar amide bond in the sTCO-S-S-sTCO compound 2.

XPS of functionalized magnetic microparticles. XPS is a surface analytical technique able to provide precise atomic level compositions of the first approximately 10 nm of a surface. Previously, XPS has been used to calculate the elemental compositions of micro- and nanoparticles on flat surfaces as well as monolayer coverage of proteins on charged surfaces and membrane environments. Here, XPS was used to follow the addition of sTCO and precise loading of tsCA₂₃₃ on amine-functionalized iron oxide microparticles (Fe beads). The XPS average atomic compositions of C₁ₛ, N₁ₛ, and Fe₂ₚ were determined for each sample type. XPS spectra were collected for each step in the sample preparation in order to track the precise loading (either 25%, 50%, or 100%) of tsCA₂₃₃ on the surface of the microparticles. Specifically, the samples are bare Fe beads, sTCO functionalized Fe beads, sTCO functionalized Fe beads with tsCA_{WT}, 25% loading of tsCA₂₃₃, 50% loading of tsCA₂₃₃, or 100% loading of tsCA₂₃₃. In all cases, except for the incubation of tsCA_{WT} and the sTCO functionalized Fe beads, the average elemental atomic compositions relating to carbon and nitrogen increased while the average atomic percent composition of iron decreased, which can be directly related to the C₁ₛ, N₁ₛ, and Fe₂ₚ core level electron. Adding sTCO adds carbon to the system and thus the detected nitrogen and iron atomic percent decreased while the carbon increased (addition of sTCO to the Fe beads). As tsCA₂₃₃ was applied, which contain carbon and nitrogen, to the microparticles, the concentration of carbon and nitrogen increased, which is directly detectable as an increase in the atomic percentage of C₁ₛ and N₁ₛ. Furthermore, adding biomolecules to the surface increases the overlayer thickness on the microparticles, increasing the attenuation of electrons from the Fe core of the microparticles, subsequently decreased the detected average atomic percent of Fe in the samples. Experimental results support these theoretical trends which suggests successful functionalization of the amine surface with sTCO and subsequent successful immobilization of tsCA₂₃₃. It is important to note that while tsCA_{WT} does not contain Tet2.0, and thus cannot react with sTCO, non-specifically bound protein leftover from the washing step, as was observed with radiolabeling and enzymatic activity, can explain the minor increase in carbon and nitrogen concomitant with slight a decrease in iron. Thus, these XPS data confirm the conversion of functionalized amine-beads to sTCO-beads and subsequently confirms the immobilization of tsCA₂₃₃.

Next, the surface load of protein at each step was determined and compared to theoretical loading amounts. It is important to normalize each sample to the starting sample of bare Fe beads so that we can determine the monolayer load of the protein on the beads. By normalizing the N₁ₛ atomic percent composition of each sample and comparing the value to published XPS data for monolayers we can estimate the load of tsCA₂₃₃. The values for the normalized N₁ₛ XPS data are: sTCO functionalized Fe beads (0.08±0.06), tsCA_{WT} (0.49±0.06), 25% tsCA₂₃₃ (2.19±0.31), 50% tsCA₂₃₃ (3.49±0.10), 100% tsCA₂₃₃ (6.36±0.12). For protein load on the sTCO-beads this normalized Nis corresponds to ˜52±1% for the “100%” tsCA₂₃₃ (50% binding capacity), about 28±1% for the “50%” tsCA₂₃₃ (25% binding capacity) load, and about 18±3% for the 25% tsCA₂₃₃ (12.5% binding capacity) load. The experimental results closely matched the theoretical loading percent compositions, and thus, protein-limited immobilization is well supported by XPS.

XPS of functionalized flat gold surfaces with thiolated sTCO. XPS was also used to follow the formation of sTCO-SAMs and immobilization of sfGFP₁₅₀ on flat gold surfaces. XPS spectra for each step was collected in the sample preparation in order to track the addition of sTCO and sfGFP₁₅₀ on the surface of the gold substrates. Specifically, the samples included bare gold, sTCO-SAM functionalized bare gold, sTCO-SAM functionalized bare gold with sfGFP₁₅₀ loaded at 1.0 μM. In all cases, the average elemental atomic compositions relating to carbon and nitrogen increased while the average atomic percent composition of gold decreased, which can be directly related to the Cis, Nis, and Au_{4f} core level electrons. Formation of sTCO-SAMs increases carbon and nitrogen in the system and thus the nitrogen detected atomic percent increased while the gold signal decreased (both nitrogen and gold). However, the carbon atomic percent appeared constant, which can be attributed to advantageous carbon present in the bare gold sample when compared to the sample with sTCO-SAM. Immobilization of sfGFP₁₅₀, which contains carbon and nitrogen, causes a directly detectable increase in the atomic percentage of these elements, while simultaneously increasing the overlayer thickness which attenuates liberated electrons from the Au surface, thereby decreasing the atomic percentage of Au in the sample. Experimental results matched these theoretical trends, which supports the successful formation of sTCO-SAMs and subsequent immobilization of sfGFP₁₅₀. Taken together, these results match the trend for immobilization of tsCA₂₃₃ onto sTCO-beads, which supports the ability of this immobilization reaction to enable protein-limited loading on different surfaces.

### Comparative Surface Immobilization of Representative Tetrazine-Modified Proteins

Immobilization of sfGFP-N150[Tet2.0] and sfGFP-N150[Tet3.0] onto sTCO-sepharose resin was compared. The present invention provides site-specific, pre determined immobilization of proteins or functional protein fragments containing either a Tet2.0 or a Te3.0 non-canonical amino acid (ncAA) onto strained trans-cyclooctene surfaces in a defined orientation at greater than 80% of the pre-determined amount, and wherein the protein or functional protein fragment retains at least 80% activity of the Tet2.0-containing protein or functional protein fragment. Tet3.0 is a structural isomer of Tet2.0.

Immobilization of sfGFP-N150[Tet2.0] and sfGFP-N150[Tet3.0] onto sTCO-sepharose resin was compared. Superfolder green fluorescent protein (sfGFP) containing either Tet2.0 and Tet3.0 at site 150 (sfGFP-N150[Tet2.0] and sfGFP-N150[Tet3.0], respectively) was generated using genetic code expansion, and was immobilized at pre-determined amounts (relative loads of “100%,” “50%,” and “25%,” respectively) onto sTCO-functionalized sepharose resin, and quantified by fluorescence.

Generation of sfGFP-N150[Tet2.0] and sfGFP-N150[Tet3.0]. Escherichia coli cells containing a machinery plasmid housing a suppression pair specific to either Tet2.0 or Tet3.0 in combination with a plasmid encoding a TAG-interrupted sfGFP at position 150 were cultured in 50 mL of auto-induction media containing 500 μM Tet2.0 or Tet3.0. Expression was allowed to induce for approximately 24 hours following initial inoculation, and the cells were harvested, lysed, and the resulting Tet2.0- or Tet3.0-containing protein purified using immobilized metal affinity chromatography, and de-salted into a PBS buffer (50 mM sodium phosphate, 100 mM sodium chloride, pH 7.0).

Generation of sTCO-Sepharose. 250 μL of NHS-activated Sepharose 4 Fast Flow resin (GE Healthcare Life Sciences) were rinsed 3 times in ultrapure water prior to resuspension in 100 mM HEPES buffer (pH 8.0) containing approximately 28.75 μmols sTCO-NH₂ and allowed to react for 90 minutes at room temperature. The resulting resin was then resuspended in a 1 M ethanolamine solution (pH 8.5) for an additional 1 hour at room temperature before being stored in methanol.

Immobilization of sfGFP-N150[Tet2.0] and sfGFP-N150[Tet3.0] in defined amounts on sTCO-Sepharose resin. sfGFP proteins containing either Tet2.0 or Tet3.0 were exposed to approximately 6.25 μL sTCO-Sepharose at defined amounts, herein defined as “100%” (250 pmols), “50%” (125 pmols), and “25%” (62.5 pmol), for approximately 5 minutes at room temperature in 50 μL of PBS (n=2). The resulting supernatant was withdrawn and diluted to 100 μL, and the resin resuspended in 100 μL of PBS buffer. Samples were then quantified, along with a reference solution (containing the amount of protein exposed to resin, herein referred to as “Free”), on a microplate reader to assess sfGFP fluorescence of each sample. The fluorescence values were then averaged across replicates, and normalized to report relative fluorescence units (RFU)

Results. As can be seen in FIGS. 7A and 7B, the majority of protein (>80%) was removed from solution upon exposure to sTCO-Sepharose (FIG. 7A), and concomitantly transferred to the sTCO-resin (FIG. 7B) in a consistent and predictable manner across all loads. Importantly, the immobilization efficiency of sfGFP-N150[Tet3.0] was not observed to differ significantly from that of sfGFP-N150[Tet2.0], indicating that Tet3.0 and Tet2.0 can be used to perform pre-determined immobilization on sTCO-functionalized surfaces to similar effect.

While illustrative embodiments have been illustrated and described, it will be appreciated that various changes can be made therein without departing from the spirit and scope of the invention.

1. A method for immobilizing a protein or functional protein fragment on a surface in a controlled orientation, comprising contacting a tetrazine-modified protein or a tetrazine-modified functional protein fragment with a trans-cyclooctene-modified surface to provide a surface having the protein or functional protein fragment immobilized thereon, wherein the tetrazine-modified protein or the tetrazine-modified functional protein fragment has been genetically encoded to include a tetrazine moiety at a predetermined amino acid site, wherein the tetrazine-modified protein or tetrazine-modified functional protein fragment is prepared by genetic encoding using a non-canonical amino acid bearing a tetrazine moiety represented by the formula: 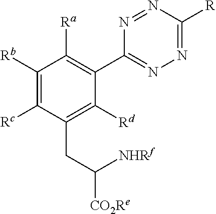
or a stereoisomer or salt thereof, wherein
R is selected from substituted or unsubstituted C1-C6 alkyl group and substituted or unsubstituted phenyl group;
R^{a}, R^{b}, R^{c}, and R^{d} are independently selected from hydrogen, C1-C3 alkyl, C1-C3 haloalkyl, C1-C3 alkoxy, and halo;
R^{e} is hydrogen, a counter ion, or a carboxyl protecting group; and
R^{f} is hydrogen or an amine protecting group.

2. The method of claim 1, wherein R is selected from substituted or unsubstituted C1-C6 alkyl group; R^{a}, R^{b}, R^{c}, and R^{d} are hydrogen; R^{e} is hydrogen, a counter ion, or a carboxyl protecting group; and R^{f} is hydrogen or an amine protecting group.

3. The method of claim 1, wherein the tetrazine-modified protein or functional protein fragment is prepared by genetic encoding using a non-canonical amino acid bearing a tetrazine moiety selected from 3-(6-methyl-s-tetrazin-3-yl)phenylalanine (Tet-v3.0-methyl), 3-(6-ethyl-s-tetrazin-3-yl)phenylalanine (Tet-v3.0-ethyl), 3-(6-isopropyl-s-tetrazin-3-yl)phenyl alanine (Tet-v3.0-isopropyl), 3-(6-t-butyl-s-tetrazin-3-yl)phenylalanine (Tet-v3.0-t-butyl), or 3-(6-n-butyl-s-tetrazin-3-yl)phenylalanine (Tet-v3.0-n-butyl).

4. A method for efficiently immobilizing a protein or functional protein fragment on a surface, comprising contacting a tetrazine-modified protein or a tetrazine-modified functional protein fragment with a trans-cyclooctene-modified surface to provide a surface having the protein or functional protein fragment immobilized thereon, wherein contacting the tetrazine-modified protein or the tetrazine-modified functional protein fragment with a trans-cyclooctene-modified surface comprises contacting a pre-determined amount of the protein or functional protein fragment and the amount of protein or functional protein fragment immobilized on the surface is at least about 80 percent of the pre-determined amount of the protein or functional protein fragment contacted with the surface.

5. A method for immobilizing a protein or functional protein fragment on a surface with retention of the activity of the protein or protein fragment, comprising contacting a tetrazine-modified protein or a tetrazine-modified functional protein fragment with a trans-cyclooctene-modified surface to provide a surface having the protein or functional protein fragment immobilized thereon, wherein the protein or functional protein fragment immobilized on the surface retains at least about 80 percent of the activity of the tetrazine-modified protein or functional protein fragment.

6. The method of claim 4, wherein contacting a tetrazine-modified protein or functional protein fragment with a trans-cyclooctene-modified surface comprises contacting a pre-determined amount of the protein or functional protein fragment and the amount of protein or functional protein fragment immobilized on the surface is about 90 percent of the pre-determined amount of the protein or functional protein fragment contacted with the surface.

7. The method of claim 5, wherein the protein or functional protein fragment immobilized on the surface retains about 100 percent of the activity of the tetrazine-modified protein or functional protein fragment.

8. The method of claim 1, wherein contacting a tetrazine-modified protein or functional protein fragment with a trans-cyclooctene-modified surface comprises contacting a pre-determined amount of the protein or functional protein fragment and the amount of protein or functional protein fragment immobilized on the surface is at least about 80 percent of the pre-determined amount of the protein or functional protein fragment contacted with the surface.

9. The method of claim 1, wherein the protein or functional protein fragment immobilized on the surface retains at least about 80 percent of the activity of the tetrazine-modified protein or functional protein fragment.

10. The method of claim 1, wherein the tetrazine-modified protein or functional protein fragment is an enzyme or functional fragment thereof, a binding protein or functional fragment thereof, or an antibody or functional fragment thereof.

11. The method of claim 1, wherein the surface is a glass surface, a metal surface, a polymer surface, or a bead surface.

12. The method of claim 4, wherein the tetrazine-modified protein or functional protein fragment is prepared by genetic encoding using a non-canonical amino acid bearing a tetrazine moiety represented by the formula: 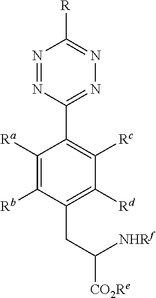
or a stereoisomer or salt thereof, wherein
R is selected from substituted or unsubstituted C1-C6 alkyl group and substituted or unsubstituted phenyl group;
R^{a}, R^{b}, R^{c}, and R^{d} are independently selected from hydrogen, C1-C3 alkyl, C1-C3 haloalkyl, C1-C3 alkoxy, and halo;
R^{e} is hydrogen, a counter ion, or a carboxyl protecting group; and
R^{f} is hydrogen or an amine protecting group.

13. The method of claim 12, wherein R is selected from substituted or unsubstituted C1-C6 alkyl group; R^{a}, R^{b}, R^{c}, and R^{d} are hydrogen; R^{e} is hydrogen, a counter ion, or a carboxyl protecting group; and R^{f} is hydrogen or an amine protecting group.

14. The method of claim 4, wherein the tetrazine-modified protein or functional protein fragment is prepared by genetic encoding using a non-canonical amino acid bearing a tetrazine moiety selected from 4-(6-methyl-s-tetrazin-3-yl)phenylalanine (Tet-v2.0-methyl), 4-(6-ethyl-s-tetrazin-3-yl)phenylalanine (Tet-v2.0-ethyl), 4-(6-isopropyl-s-tetrazin-3-yl)phenylalanine (Tet-v2.0-isopropyl), or 4-(6-butyl-s-tetrazin-3-yl)phenylalanine (Tet-v2.0-n-butyl).

15. The method of claim 4, wherein the tetrazine-modified protein or functional protein fragment is prepared by genetic encoding using a non-canonical amino acid bearing a tetrazine moiety represented by the formula: 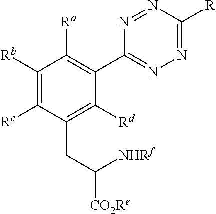
or a stereoisomer or salt thereof, wherein
R is selected from substituted or unsubstituted C1-C6 alkyl group and substituted or unsubstituted phenyl group;
R^{a}, R^{b}, R^{c}, and R^{d} are independently selected from hydrogen, C1-C3 alkyl, C1-C3 haloalkyl, C1-C3 alkoxy, and halo;
R^{e} is hydrogen, a counter ion, or a carboxyl protecting group; and
R^{f} is hydrogen or an amine protecting group.

16. The method of claim 15, wherein R is selected from substituted or unsubstituted C1-C6 alkyl group; R^{a}, R^{b}, R^{c}, and R^{d} are hydrogen; R^{e} is hydrogen, a counter ion, or a carboxyl protecting group; and R^{f} is hydrogen or an amine protecting group.

17. The method of claim 4, wherein the tetrazine-modified protein or functional protein fragment is prepared by genetic encoding using a non-canonical amino acid bearing a tetrazine moiety selected from 3-(6-methyl-s-tetrazin-3-yl)phenylalanine (Tet-v3.0-methyl), 3-(6-ethyl-s-tetrazin-3-yl)phenylalanine (Tet-v3.0-ethyl), 3-(6-isopropyl-s-tetrazin-3-yl)phenylalanine (Tet-v3.0-isopropyl), 3-(6-t-butyl-s-tetrazin-3-yl)phenylalanine (Tet-v3.0-t-butyl), or 3-(6-n-butyl-s-tetrazin-3-yl)phenylalanine (Tet-v3.0-n-butyl).

18. A surface having a protein or functional protein fragment immobilized thereon obtainable by the method of claim 1.

19. A surface having a protein or functional protein fragment immobilized thereon, comprising a protein or functional protein fragment covalently coupled a surface,
wherein the protein or functional protein fragment is a tetrazine-modified protein or a tetrazine-modified functional protein fragment, and wherein the tetrazine-modified protein or the tetrazine-modified functional protein fragment has been genetically encoded to include a tetrazine moiety at a predetermined amino acid site,
wherein the surface is a trans-cyclooctene-modified surface, and
wherein the protein or functional protein fragment is covalently coupled to the surface via the reaction of the tetrazine of the tetrazine-modified a protein or functional protein fragment with the trans-cyclooctene of the trans-cyclooctene-modified surface.

20. A method for measuring the binding of a ligand to a protein or functional protein fragment, comprising:
contacting a ligand with a surface of claim 18; and
determining whether the ligand binds to the protein or functional protein fragment immobilized on the surface.

21. The method of claim 20, wherein measuring the binding of a ligand to a protein or functional protein fragment is a screening process useful in therapeutic drug discovery.
